(19) United States
(12) Patent Application Publication
(10) Pub. No.: US 2025/0308663 A1
(43) Pub. Date: Oct. 2, 2025

(54) INTEGRATION OF RADIOLOGIC, PATHOLOGIC, AND GENOMIC FEATURES FOR PREDICTION OF RESPONSE TO IMMUNOTHERAPY

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventors: Jianjiong Gao, New York, NY (US); Sohrab Shah, New York, NY (US); Rami Vanguri, New York, NY (US); Andrew Aukerman, New York, NY (US); Matthew Hellmann, New York, NY (US); Jennifer Sauter, New York, NY (US); Natally Horvat, New York, NY (US); Michelle Ginsburg, New York, NY (US); Andrew Pagano, New York, NY (US); Jose de Arimateia Batista Araujo-Filho, New York, NY (US); Jia Luo, New York, NY (US); Jacklynn Egger, New York, NY (US)

(21) Appl. No.: 18/863,287

(22) PCT Filed: May 5, 2023
(86) PCT No.: PCT/US2023/021178

§ 371 Date: Nov. 5, 2024

Related U.S. Application Data
(60) Provisional application No. 63/339,081, filed on May 6, 2022.

(51) Int. Cl. G16H 20/10; G06T 7/00; G16B 20/20; G16H 15/00; G16H 30/40; G16H 50/20; G16H 50/30; G16H 50/70

(52) U.S. Cl. CPC G16H 20/10; G06T 7/0012; G16B 20/20; G16H 15/00; G16H 30/40; G16H 50/20; G16H 50/30; G16H 50/70; G06T 2207/10072

(57) ABSTRACT

Presented herein are systems, methods, and non-transient computer readable media for determining predicted response scores of subjects. A computing system may identify a first feature set for a first subject to be administered with immunotherapy to address a condition. The first feature set may include one or more of: (i) a first radiological feature identified in a tomogram of a section associated with the condition in the first subject, (ii) a first immunohistochemistry (IHC) feature derived from an image of a sample associated with the first subject, and (iii) a first genomic feature obtained from gene sequencing of the first subject for genes associated with the condition. The computing system may apply the first feature set to a model. The computing system may determine, from applying the first feature set to the model, a predicted score identifying a response to the immunotherapy to be administered to the first subject.

### CROSS-REFERENCE TO RELATED PATENT APPLICATIONS

The present application claims priority to U.S. Provisional Patent Application No. 63/339,081, titled “Integration of Radiologic, Pathologic, and Genomic Features for Prediction of Response to Immunotherapy,” filed May 6, 2022, which is incorporated herein by reference in its entirety.

### BACKGROUND

A computing system may apply various machine learning (ML) techniques on an input to generate an output.

### SUMMARY

Aspects of the present disclosure are directed to systems, methods, and non-transient computer readable media for determining predicted response scores of subjects. A computing system may identify a first feature set for a first subject to be administered with immunotherapy to address a condition. The first feature set may include one or more of: (i) a first radiological feature identified in a tomogram of a section associated with the condition in the first subject, (ii) a first immunohistochemistry (IHC) feature derived from an image of a sample associated with the first subject, and (iii) a first genomic feature obtained from gene sequencing of the first subject for genes associated with the condition. The computing system may apply the first feature set to a model comprising a set of weights. The set of weights for the model may be established using (i) a plurality of second feature sets from a respective plurality of second subjects and (ii) a plurality of expected scores each identifying a respective response to immunotherapy in corresponding second subject of the plurality of second subjects. The computing system may determine, from applying the first feature set to the model, a predicted score identifying a response to the immunotherapy to be administered to the first subject. The computing system may store, using one or more data structures, an association between the first subject and the predicted score identifying the response.

In some embodiments, the computing system may determine that at least one feature of the first feature set corresponding to the first radiological feature, the first IHC feature, and the first genomic feature is unavailable. In some embodiments, the computing system may assign a defined value to the at least one feature in the first feature set, responsive to determining that the at least one feature is unavailable. In some embodiments, the computing system may apply the first feature set comprising the at least one feature assigned to the defined value.

In some embodiments, the computing system may determine that all of features corresponding to the first radiological feature, the first IHC feature, and the first genomic feature of the first feature set are available. In some embodiments, the computing system may maintain the first feature set responsive to determining that all the features in the first feature set are available. In some embodiments, the computing system may classify the first subject into one of a plurality of response groups based on a comparison between the predicted score identifying a likelihood of improvement from the immunotherapy and a threshold for each of the plurality of response groups.

In some embodiments, the computing system may determine a plurality of risk scores for the predicted score, the plurality of risk scores identifying: (i) a first score corresponding to the first radiological feature, (ii) a second score corresponding to the first IHC feature, and (iii) the first genomic feature. In some embodiments, the computing system may generate a survival function identifying the predicted score for the response to the immunotherapy by the first subject over a time period. In some embodiments, the computing system may provide information based on the association between the first subject and the predicted score identifying the response.

In some embodiments, the first radiological feature may be based on a region of interest (ROI) identified in the tomogram corresponding to a portion of the section associated with the condition to be addressed with the immunotherapy. In some embodiments, the first IHC feature derived from the image may be based on a gray level co-occurrence matrix (GLCM) autocorrelation matrix correlated with at least one of a tumor proportion score (TPS) or a progression-free survival (PFS) measure. In some embodiments, the first genomic feature may identify one or more genes associated with therapy response comprising at least one of: (i) an altered oncogene, (ii) an altered tumor suppressor, or (iii) an altered transcription regulator.

### BRIEF DESCRIPTION OF THE DRAWINGS

FIGS. 1A-E Multimodal cohort characteristics and schema outlining the project. A Multimodal cohort heatmap listing clinical, pathological, radiomic, and genomic characteristics. B Lung cancer histology breakdown. C Distributions of PD-L1 tumor proportion score, tumor mutational burden, and number of annotated lesions between responders (PR/CR) and non-responds (SD/PD). D Analysis overview using DyAM to integrate multiple modalities to predict immunotherapy response. E Train-test-validate breakdown and optimization scheme.

FIGS. 2A-D Extraction of CT radiomics features and association with response. A Radiomics feature extraction pipeline using expert segmented thoracic CT scans. Superpixel-based perturbations on original segmentations used for feature selection. B Expert CT segmentation examples including lung parenchymal (PC) (top), pleural (PL) (middle) and lymph node lesions (LN) (bottom), with the original image, segmentation, and randomized contour example. C Principal component decomposition of radiomics features, including superpixel-based perturbations. D Response prediction performance using logistic regression classifiers (LR) for each type of lesion as well as averaging-based patient-level prediction aggregation by averaging (LR Rad-Average) outcomes across all lesions and the multiple instance learning model (MILR). Results with AUC<0.5 are not shown. Error bars represent the 95% CI on the AUC value based on DeLong's method.

FIGS. 3A-E PD-L1 immunohistochemistry (IHC) feature derivation and prediction of response. A Analysis pipeline to extract image-based IHC-texture starting from scanned PD-L1 IHC slides. B Normalized-value distributions of GLCM and pixel intensity derived image features stratified by response for the best performing summary statistic in each GLCM class. Featured indicated by the red asterisk emerged as salient features in the logistic regression (LR) fit. C Representative PD-L1 IHC slides corresponding to the maximum (top), median (middle) and minimum (bottom) of the GLCM autocorrelation distribution, with a low power, high power, stain intensity, and pixel-wise GLCM sample patches. D Correspondence of the example GLCM features in C between low, medium and high bins of TPS. E Response prediction performance using LR classifiers with PD-L1 features including TPS (LR PDL1-TPS), pixel and GLCM autocorrelation image features (LR IHC-A), only the complete GLCM features (LR IHC-G), and the result of aggregating patient-level predictions by averaging classifier outcomes from LR IHC-A, IHC-G and LR PDL1-TPS (LR Path-Average). Error bars represent the 95% CI on the AUC value.

FIGS. 4A-C Modeling of response from genomic alterations and TMB. A Adjusted hazard ratios using Cox proportional hazard model analysis of genomic variables alongside single feature AUCs. B Comparison of EGFR and STK11 feature coefficients with and without the inclusion of TMB in the model. Boxes represent interquartile range. C AUCs resulting from models using: only TMB, genomic alterations (without TMB), averaging predictions from the TMB and alterations models, and fitting a model with both TMB and genomic alterations.

FIGS. 5A-C Dynamic Attention with Masking (DyAM) based unimodal and multimodal prediction of response. A DyAM was used for multimodal integration. CT segmentation-derived features were separated by lesion type (lung parenchymal, pleural, lymph node) with separate attention weights applied. Attention weights are also used for genomics and PD-L1 IHC derived features to result in a final prediction of response. The model's modality specific risk score, attention scores, and overall score can be analyzed. B Overall score analysis: Kaplan-Meier survival analysis using DyAM to integrate all three modes of data results in significant separation of responders from non-responders. C Response predictions summary plot with combinations of input data modalities using DyAM and logistic regression models. The coarse, red hatched regions represent the 1-sigma error on the permutation tested AUC measurement, and the fine, grey hatched regions represent the 1-sigma error from repeated subsampling. Error bars represent 95% confidence intervals from the merged scores and stars correspond to 1-4+ sigma separation of the observed and permutation tested values.

FIG. 6A-C DyAM-based multi-modal analysis. A Hazard ratios and single feature AUCs of the covariates, (Left) overall risk score analysis: forest plot of the DyAM model score with respect to other clinical variables, (Right) partial risk-score analysis: forest plot of the modality specific risk-scores from logistic regression compared to DyAM. The vertical dashed lines represent a null hazard ratio. B A zoom in of the first 12 months separated by quartile showing separation of early progression events. C Alpine plots comparing the overall model score after reweighting the input modalities as a function of a multiplier to a single modality's attention, for the model's AUC (left), hazard ratio (middle), and PFS ratio at 4 months in the lower and higher quartiles (right).

FIG. 7 Cohort Venn diagram.

FIG. 8A-E 10-fold cross validation receiver operating characteristic (ROC).

FIG. 9A-E Sub-sampling receiver operating characteristic (ROC).

FIG. 10 F1 scores across radiology, pathology, genomics DyAM unimodal, DyAM biomodal, DyAM multi-modal, and DyAM multi-modal with tumor proportion scores (TPS).

FIG. 11 Precision scores across radiology, pathology, genomics DyAM unimodal, DyAM biomodal, DyAM multi-modal, and DyAM multi-modal with tumor proportion scores (TPS).

FIG. 12 Recall scores across radiology, pathology, genomics DyAM unimodal, DyAM biomodal, DyAM multi-modal, and DyAM multi-modal with tumor proportion scores (TPS).

FIG. 13 logistic regression (LR) clinical coefficients.

FIG. 14 logistic regression (LR) radiomic lung parenchymal (Rad-PC) coefficients.

FIG. 15 Logistic regression (LR) radiomic pleural (Rad-PL) coefficients.

FIG. 16 Logistic regression (LR) radiomic lymph node (Rad-LN) coefficients.

FIG. 17 Logistic regression (LR) immunohistochemistry pixel and gray-level co-occurrence matrix (GLCM) autocorrelation image features (IHC-A) coefficients.

FIG. 18 Logistic regression (LR) immunohistochemistry (GLCM) autocorrelation image features (IHC-G) coefficients.

FIG. 19 Logistic regression (LR) genomics coefficients.

FIGS. 20A-H Risk vectors for DyAM RAD, IHC-G, and genomics model weights.

FIGS. 21A-D Risk vectors for DyAM RAD, IHC-A, genomics, and PDL1 model weights.

FIG. 22 depicts a block diagram of a system for determining predicted responses scores of subjects using multimodal models in accordance with an illustrative embodiment.

FIG. 23A depicts a block diagram of a process of extracting multimodal features in the system for determining predicted responses in accordance with an illustrative embodiment.

FIG. 23B depicts a block diagram of a process of applying response prediction models to multimodal features in accordance with an illustrative embodiment.

FIG. 24 depicts a flow diagram of a method of determining predicted responses using multimodal feature sets in accordance with an illustrative embodiment.

FIG. 25 depicts a block diagram of a server system and a client computer system in accordance with an illustrative embodiment.

### DETAILED DESCRIPTION

Following below are more detailed descriptions of various concepts related to, and embodiments of, systems and methods for. It should be appreciated that various concepts determining predicted response scores of subjects introduced above and discussed in greater detail below may be implemented in any of numerous ways, as the disclosed concepts are not limited to any particular manner of implementation. Examples of specific implementations and applications are provided primarily for illustrative purposes.

Section A describes multi-modal integration of radiology, pathology, and genomics for prediction of response to PL-(L)1 blockade in patients with non-small cell lung cancer;

Section B describes systems and methods of determining predicted response scores of subjects using multimodal models; and

Section C describes a network environment and computing environment which may be useful for practicing various embodiments described herein.

A. Multi-Modal Integration of Radiology, Pathology, and Genomics for Prediction of Response to PD-(L)1 Blockade in Patients with Non-Small Cell Lung Cancer

Guided by domain expert annotations, a computational workflow may be developed to extract discriminative data elements for each patient and trained an attention-gated machine learning approach to integrate the multimodal features into a risk prediction model. Integrating radiology, pathology, genomic, and clinical features in a multimodal model outperformed unimodal measures, including tumor mutational burden and PD-L1 IHC score.

Immunotherapy is used to treat almost all patients with advanced non-small cell lung cancer (NSCLC). However, identifying robust biomarkers to predict treatment response remains challenging. The predictive capacity of integrating medical imaging, histopathologic, and genomic features were evaluated as a new class of multimodal biomarker for immunotherapy response. A cohort of 247 patients with advanced NSCLC were examined with multimodal baseline data obtained during diagnostic clinical workup, including CT scan images and digitized PD-L1 immunohistochemistry (IHC) slides, and known outcomes to immunotherapy. Guided by domain expert annotations, a computational workflow may be developed to extract discriminative data elements for each patient and trained an attention-gated machine learning approach to integrate the multimodal features into a risk prediction model. Integrating radiology, pathology, genomic, and clinical features in a multimodal model (AUC=0.80, 95% CI 0.74-0.86) outperformed unimodal measures, including tumor mutational burden (AUC=0.61, 95% CI 0.52-0.70) and PD-L1 IHC score (AUC=0.73, 95% CI 0.65-0.81). This approach therefore provides a quantitative rationale for using multimodal features to improve prediction of immunotherapy response over standard of care approaches in patients with NSCLC using expert-guided machine learning

### 1. Introduction

Immunotherapies blocking programmed cell death protein 1 (PD-1) and its ligand (PD-L1) to activate and reinvigorate cytotoxic anti-tumor T-cells have rapidly altered the treatment landscape of non-small cell lung cancer (NSCLC). In just four years, the PD-1/PD-L1 pathway blockade (abbreviated as PD-(L)1) has become a routine component of treatment for nearly all patients. These treatments represent potential for long-term, durable benefit for a subset of individuals with advanced lung cancer. Recent reports estimate the five-year survival of patients on the first clinical trials at 10-15%. PD-(L)1 blockade is now being tested in earlier stages of lung cancer and in combination with other therapies.

This shift in treatment has highlighted the need to identify predictors of response to immunotherapy. Multiple independent analyses have pinpointed individual baseline clinical features as potential independent predictors of response (e.g., antibiotic use, systemic steroid use⁴, the neutrophil-to-lymphocyte ratio at diagnosis), individual genomic alterations (such as mutations in EGFR and STK11), and the presence of intratumoral cytotoxic T-cell populations. There are only FDA approved predictive biomarkers for immunotherapy in NSCLC: tumor PD-L1 expression assessed by immunohistochemistry (IHC) and tumor mutation burden (TMB). However, they are only modestly helpful. For example, PD-L1 expression only modestly distinguished long term response in the 5-year overall survival report of Keynote-001.

While there have been attempts to develop multimodal genomic predictive biomarkers, it is sought to develop a model which integrates and synthesizes multimodal data routinely obtained during clinical care to predict response to immunotherapy. Patients diagnosed with advanced NSCLC undergo standard-of-care tests which generate valuable data such as PD-L1 expression patterns in diagnostic tumor biopsies and radiological computed tomography (CT) images used in the staging of lung cancer. The raw data from these modalities are amenable to automated feature extraction with machine learning and image analysis tools. Accordingly, machine learning based integration of these modalities represents an opportunity to advance precision oncology for PD-(L)1 blockade by computing patient specific risk scores. Other approaches on automated deep learning methods to predict immunotherapy outcomes from CT scans has shown predictive capacity from specific lesion types. One approach uses CT scans, laboratory data and clinical data to predict NSCLC immunotherapy outcomes, but only incorporates EGFR and KRAS mutational status. However, in general the relative predictive capacity of the unimodal histology, radiology, genomic and clinical features compared to an integrated model remains poorly understood. This is in part due to a lack of datasets with multiple modalities available from the same set of patients from which systematic evaluation can be undertaken. Presented herein is a multidisciplinary study on a rigorously curated multimodal cohort of 247 NSCLC patients treated with PD-(L)1 blockade to develop DyAM, a deep learning model to predict immunotherapy response. Also presented herein is a quantitative evaluation and predictive capacity of an adaptively weighted multimodal approach relative to the unimodal features derived from histology, radiology, genomics and standard of care approved biomarkers.

### 2. Results

A. Clinical Characteristics of Patients with NSCLC Who Received PD-(L)1 Blockade

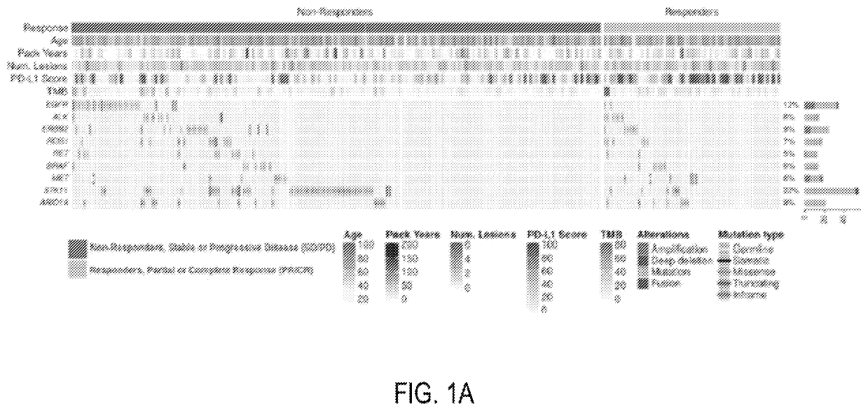

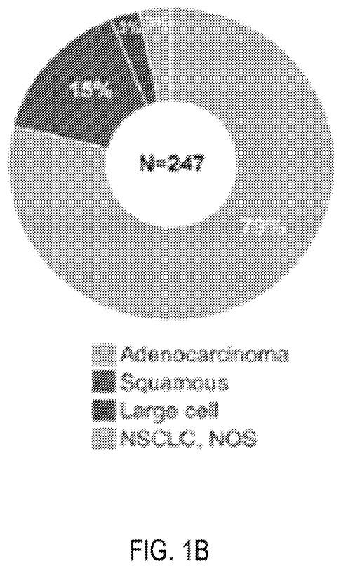

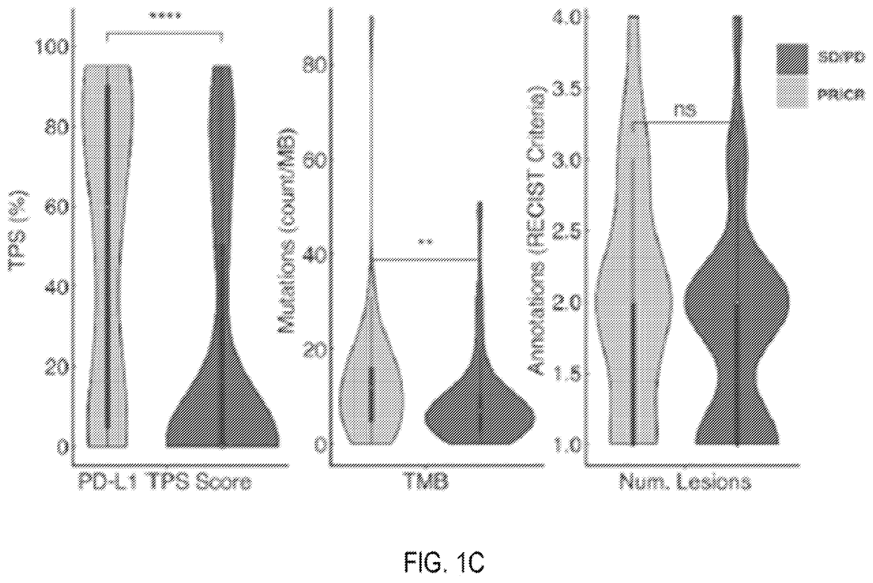

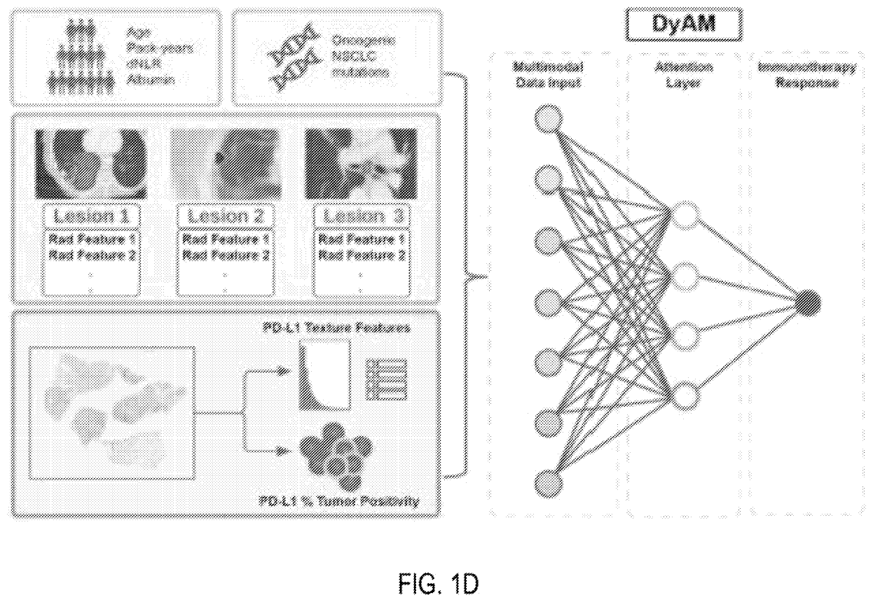

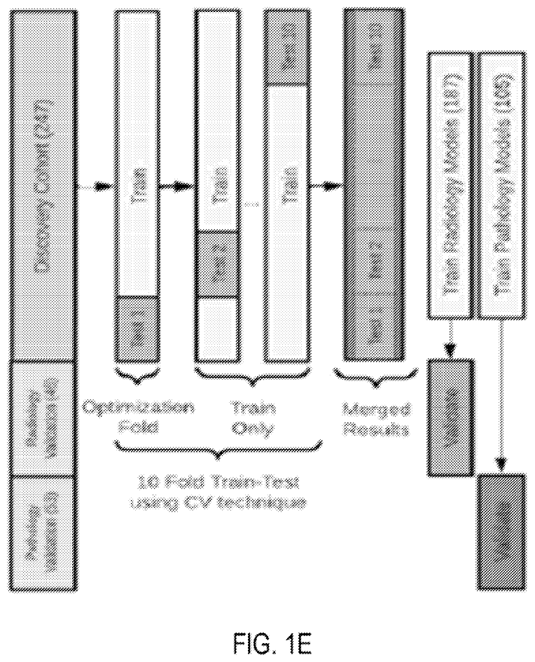

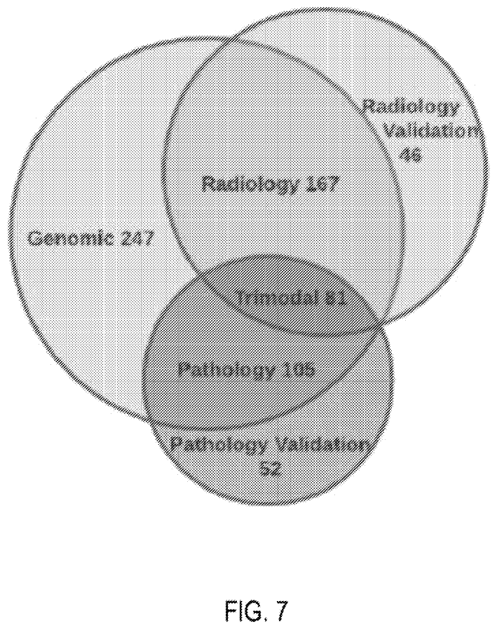

247 patients at Memorial Sloan Kettering Cancer Center (MSK) with advanced NSCLC who received PD-(L)1 blockade-based therapy between 2014-2019 (Table 1, FIG. 1A, FIG. 7), referred to as the multimodal cohort, were identified. As only 25% of the cohort responded to immunotherapy, class balancing may be consistently used in the predictive models. The multimodal cohort (Table 1) was 54% female with median age of 68 years (range=38-93 years). Overall, 218 (88%) patients had a history of smoking cigarettes (median 30 pack-years, range 0.25-165). Histological subtypes of NSCLC included 195 (79%) adenocarcinomas, 37 (15%) squamous cell carcinomas, 7 (3%) large cell carcinoma, and 8 (3%) NSCLC, not otherwise specified (NOS) (FIG. 1B). Collectively, 169 (68%) patients received one or more lines of therapy prior to starting PD-(L)1 blockade, while 78 (32%) patients received PD-(L)1 blockade as first line therapy, of which 14 (6%) received therapy in the context of a clinical trial.

Best overall response to PD-(L)1 blockade was retrospectively assessed by thoracic radiologists with RECIST (v1.1) criteria resulting in 137 (55%) patients with progressive disease (PD), 48 (20%) with stable disease (SD), 56 (23%) with partial response (PR), and 6 (2%) with complete response (CR). In this analysis, the cohort was binarized as responders (CR/PR) and non-responders (SD/PD), resulting in median progression free survival and overall survival of 2.7 months (95% CI 2.5, 3.0) and 11.4 months (95% CI 10.3-12.8), respectively.

Two additional cohorts were assembled to validate unimodal features extracted from radiological and histological data, referred to as the radiology (n=50) and pathology (n=52) cohorts, respectively (Table 1). Patients in these two cohorts did not meet the inclusion criteria for the multimodal cohort due to missing data from one of the other modalities. For example, patients would be included in the pathology cohort if they had missing radiology data due to being referred from another institution.

TABLE 1
Patient characteristics of the three orthogonal cohorts
used in this study: multimodal, radiology and pathology.

| | Training | Radiology | | Pathology | |
|---|---|---|---|---|---|
| | (n = 247) | (n = 50) | | (n = 52) | |
| Characteristics | n(%) | n(%) | | n(%) | |
| Age, media (range) | 68 (38-93) | 67 | (45-86) | 71 | (30-89) |
| Sex | | | | | |
| Male | 113 (46) | 24 | (48) | 23 | (44) |
| Female | 134 (54) | 26 | (52) | 29 | (56) |
| Performance Status | | | | | |
| ECOG 0/1 | 222 (90) | 25 | (50) | 49 | (94) |
| ECOG 2+ | 25 (10) | 25 | (50) | 3 | (6) |
| Smoking status | | | | | |
| Current/former | 218 (88) | 38 | (76) | 47 | (90) |
| Never | 29 (12) | 12 | (24) | 5 | (10) |
| Histology | | | | | |
| Adenocarcinoma | 195 (79) | 39 | (78) | 32 | (62) |
| Squamous | 37 (15) | 8 | (16) | 11 | (21) |
| Large cell | 7 (3) | 2 | (4) | 0 | (0) |
| NSCLC, NOS | 8 (3) | 1 | (2) | 9 | (17) |
| Line of Therapy | | | | | |
| 1 | 78 (32) | 5 | (10) | 35 | (67) |
| 2 | 136 (55) | 24 | (48) | 11 | (21) |
| ≥3 | 33 (13) | 21 | (42) | 6 | (12) |
| Therapy type | | | | | |
| ANTI-PD-(L1) monotherapy | 235 (95) | 48 | (96) | 52 | (100) |
| Anti-PD(L)1 + CTLA-4 | 12 (5) | 2 | (4) | 0 | (0) |
| combination | | | | | |
| PDF-L1 expression | | | | | |
| 0 | 114 (46) | — | | 0 | (0) |
| 1%-49% | 51 (21) | — | | 13 | (25) |
| ≥50% | 82 (33) | — | | 39 | (75) |
| Tissue site | | | | | |
| Lung | 109(44) | — | | 25 | (48) |
| Pleura/pleural fluid | 19 (8) | — | | 1 | (2) |
| Lymph node | 45 (18) | — | | 10 | (19) |
| Liver | 11 (4) | — | | 2 | (4) |
| Bone | 16 (7) | — | | 3 | (6) |
| Adrenal | 11 (4) | — | | 1 | (2) |
| Other | 36 (15) | — | | 10 | (19) |
| Best overall response | | | | | |
| CR/PR | 62(25) | 11 | (22) | 14 | (27) |
| SD/PD | 185 (75) | 39 | (78) | 38 | (73) |
| Tumor mutation burden | | (n = 31) | | (n = 38) | |
| ≥10 mut/Mb | 155 (63) | 9 | (29) | 22 | (58) |
| <10 mut/Mb | 92 (37) | 22 | (71) | 16 | (42) |

### B. Establishing a NSCLC Multimodal Cohort for Multimodal Predictors of ICB Response

Standard clinical biomarkers including PD-L1 tumor proportion score (TPS) and TMB were significantly different between responders and non-responders in the multimodal cohort. However, classification models using these features were unable to completely separate the two groups (TPS AUC=0.73 95% CI 0.65-0.81, TMB AUC=0.61 95% CI 0.52-0.69). (FIG. 1C). Thus, a multimodal data resource may be established by collating routinely collected clinical information, CT scans, digitized PD-L1 IHC in tissue containing NSCLC, and genomic features from the MSK-IMPACT clinical sequencing platform. These data may be used to establish a multimodal biomarker. First, the predictive capacity of each modality individually may be quantified, prior to assembling all available data into a multimodal biomarker to build an algorithm predictive of response (FIG. 1D). A 10-fold cross-validation may be performed to obtain model predictions for the entire multimodal cohort by merging results from the test-sets (FIG. 1E). The imbalance of responders and non-responders was handled by reweighting the non-responders by the ratio of class instances (0.34).

C. Unimodal Features from CT Scans Only Modestly Separates PD-(L)1 Blockade Response

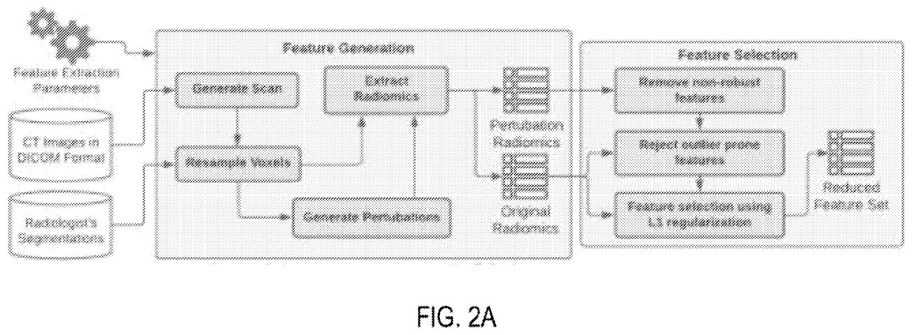

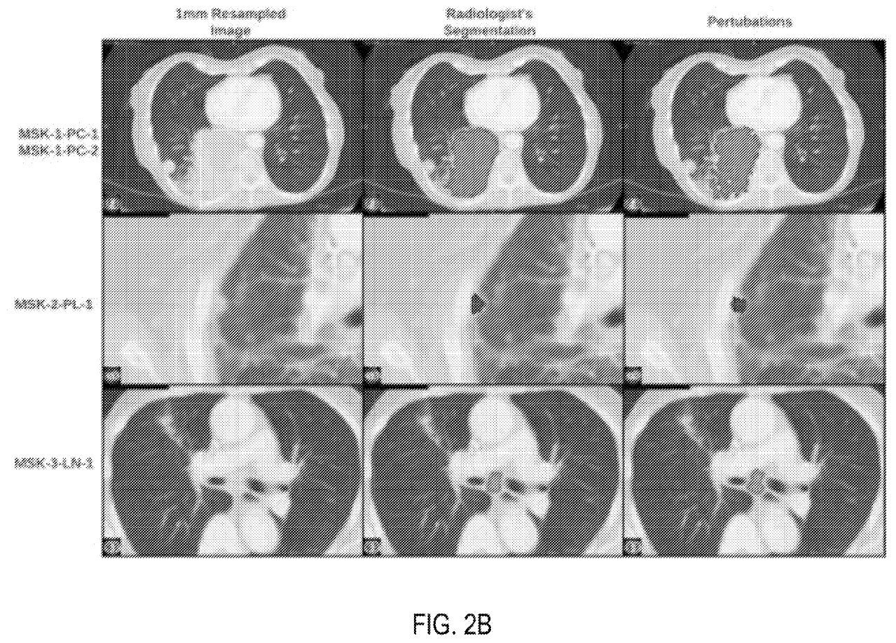

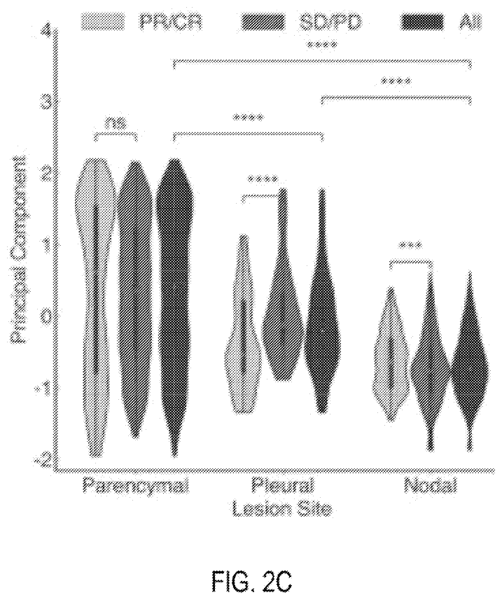

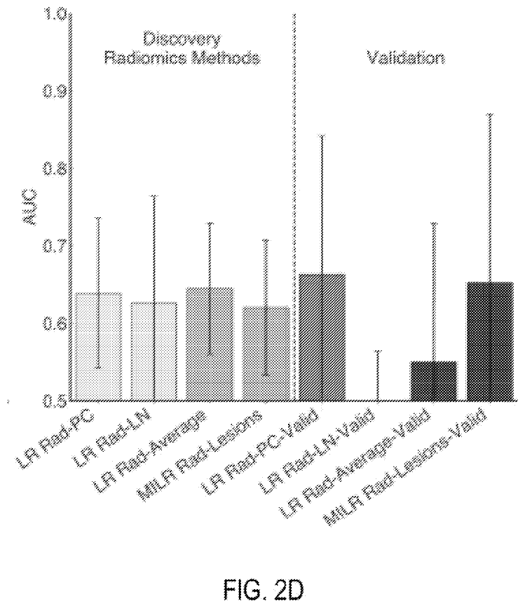

Of the 247 patients, 187 (76%) patients had disease which was clearly delineated and separable from adjacent organs. The 187 patients included 163 (87%) with lung parenchymal lesions, 21 (11%) with pleural lesions, and 67 (36%) with pathologically enlarged lymph nodes. For each patient, up to 6 lesions were segmented and site annotated by three board-certified thoracic radiologists (NH, AP, and AA). To ensure consistency in CT imaging protocols, the analysis was limited to chest imaging. The mean segmented volume for lung parenchymal, pleural, and nodal lesions was 24 (range 0.14-50, IQ 15-48), 12 (range 0.31-209, IQ 16-26), and 9.4 (range 0.82-42, IQ 37-67) cm³. This analysis pipeline (FIG. 2A) extracts robust features from the original radiologist segmentations which were augmented by superpixel-based perturbations (FIG. 2B). Principal component analysis (PCA) of all radiomics features of the original and perturbed segmentations (FIG. 2C) showed lesion-wise similarity, indicating broad preservation of the underlying texture, and significant differences in the principal component by lesion type. The similarity of radiomics features by lesion type across patients motivated building site-specific classification models. Logistic regression modeling with L1 regularization selected an average of 35, 10, and 25 features from lung parenchymal, pleural, and nodal lesions, respectively, which were used for downstream prediction of immunotherapy response (FIG. 2D). The logistic model built from features derived from pleural nodules alone was unsuccessful outside of training data (AUC=0.28, 95% CI 0.04-0.52) compared to lung parenchymal nodules (AUC=0.64, 95% CI 0.54-0.74) and pathologically enlarged lymph nodes (AUC=0.63, 95% CI 0.49-0.77). The model based on enlarged lymph nodes did not converge well, with over 20% of models performing worse than random chance with repeated sub-sampling. The average individual lesion predictions may be aggregated to construct patient-level response predictions which resulted in an overall AUC=0.65, 95% CI 0.57-0.73. An alternate model which analyzed all lesions from each patient without separation into categories may be also developed using multiple-instance learning, which resulted in similar, albeit lower, performance (AUC=0.61, 95% CI 0.52-0.70).

CT-based predictions of response were validated in the radiology cohort, consisting of 50 patients (Table 1) with expert segmentation, resulting in 40 lung parenchymal lesions, 8 pleural lesions and 22 enlarged lymph nodes. The predictive ability from features extracted from the lung parenchymal lesions (AUC=0.66, 95% CI 0.48-0.84) was consistent with the multimodal cohort (AUC=0.64, 95% CI 0.54-0.74), as were the averaging (AUC=0.55, 95% CI 0.37-0.73) and MILR based aggregation models (AUC=0.65, 95% CI 0.44-0.87). Taken together, discriminating clinical endpoints by CT derived features was modest, and primarily driven by texture in the lung parenchymal lesions. However, lesion specific feature extractions were propagated for use in the multimodal model, where relative contributions to predictive capacity were evaluated.

D. Automated PD-L1 Texture Features from Digitized Slides Approximate Pathologist Assessments

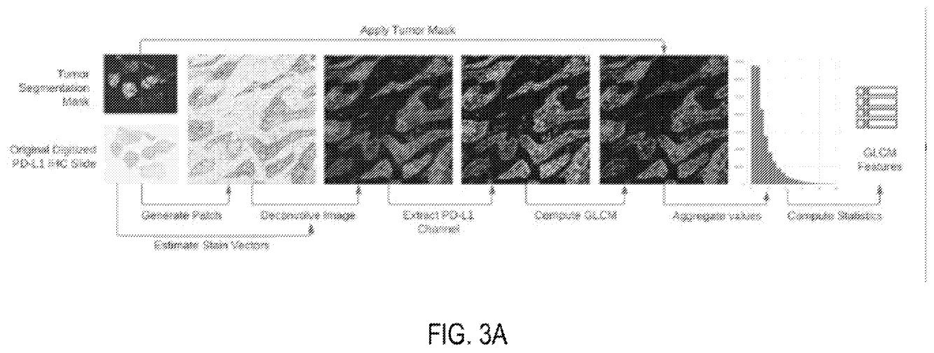

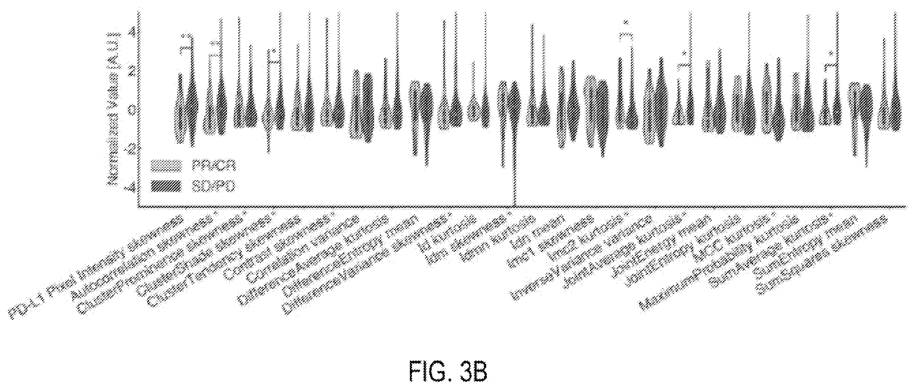

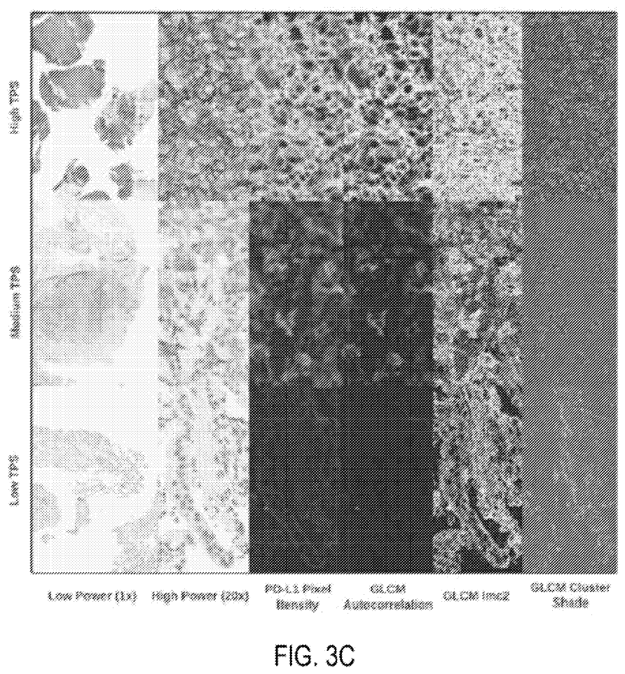

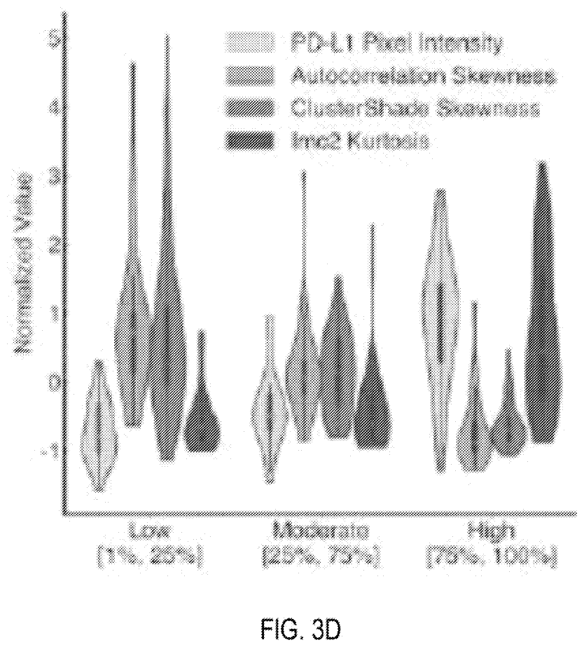

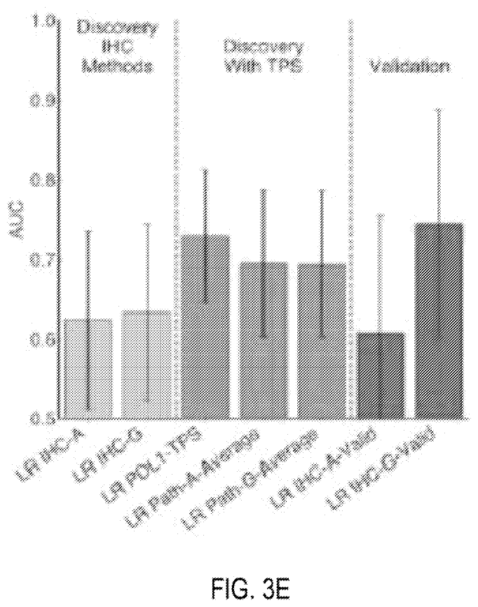

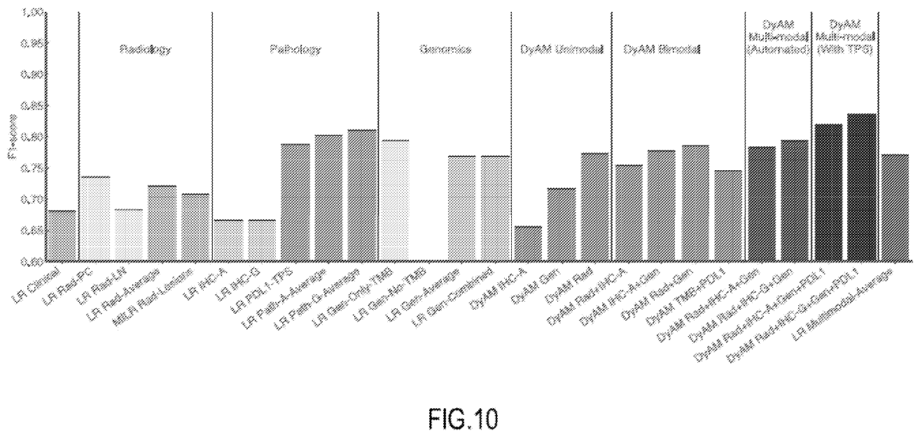

Digitized formalin-fixed paraffin-embedded (FFPE) slides of pre-treatment PD-L1 IHC performed on tumor specimens meeting quality control standards (n=201 patients (81%) may be examined. 105 (52%) tumor slides showed positive PD-L1 IHC staining (TPS≥1%) and were used to extract IHC-texture, a novel characterization of PD-L1 IHC based on the spatial distribution of expression (FIG. 3A). IHC-texture was composed of features with a wide range of statistical association to immunotherapy response (FIG. 3B). The most predictive feature, skewness of the Gray-Level Co-Occurence Matrix (GLCM) autocorrelation matrix, correlated with both TPS and PFS (AUC=0.68, r2=−0.38, r2=0.17 N=105). The maximum, median, and minimum of autocorrelation (a measure of the coarseness of the texture of an image) skewness corresponded broadly with PD-L1 IHC stain intensity as well as with contrast and edges in PD-L1 intensity between cells (FIG. 3C). Additional significant features from the logistic regression fit included cluster shade skewness and Imc2 kurtosis, which were less sensitive to the overall PD-L1 stain intensity (FIG. 3C). Furthermore, distributions of GLCM autocorrelation were statistically significantly and inversely associated with pathologist-assessed PD-L1 TPS (FIG. 3D) indicating automated feature extraction with IHC-texture could approximate expert thoracic pathologist assessment. Quantitative analysis using logistic regression modeling using 18 features based on the autocorrelation matrix and statistics of the pixel intensity distribution (IHC-A) resulted in prediction accuracy of AUC=0.62 (95% CI 0.51-0.73) which was comparable to lesion-wide radiological averaging (AUC=0.65, 95% CI 0.57-0.73), but inferior to the pathologist-assessed PD-L1 TPS (AUC=0.73, 95% CI 0.65-0.81) (FIG. 3E). While including TPS and IHC-A features reduced the AUC (FIG. 3E), other classifier metrics including accuracy, recall and F1-score increased (FIG. 10, 6 and Table 2). Including all 150 GLCM features (IHC-G) resulted in a prediction accuracy of AUC=0.63 (95% CI 0.52-0.74).

Using the IHC-A feature set, these findings may be validated in the pathology cohort, which consisted of 52 patients with positive PD-L1 expression. The result was consistent with the multimodal cohort (multimodal cohort: AUC=0.61, 95% CI 0.46-0.76 vs pathology cohort: AUC=0.62, 95% CI 0.51-0.73). GLCM autocorrelation (FIG. 3C) mean (multimodal cohort: AUC=0.67, 95% CI 0.56-0.78 vs pathology cohort: AUC=0.72, 95% CI 0.58-0.86) and skewness (multimodal cohort: AUC=0.69, 95% CI 0.58-0.80 vs pathology cohort: AUC=0.74, 95% CI 0.60-0.88) were also consistent with the multimodal cohort. The model based on the full set of GLCM features (IHC-G) had higher performance in the pathology cohort with AUC=0.74 (95% CI 0.67-0.81).

E. Genomic Predictors of Response from Clinical Sequencing Data

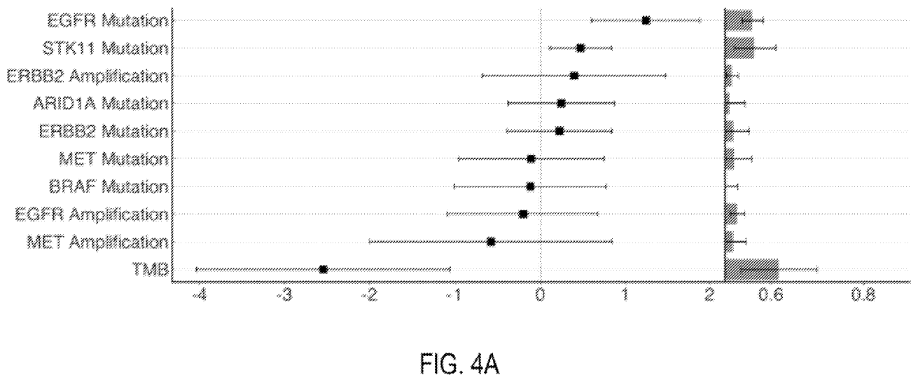

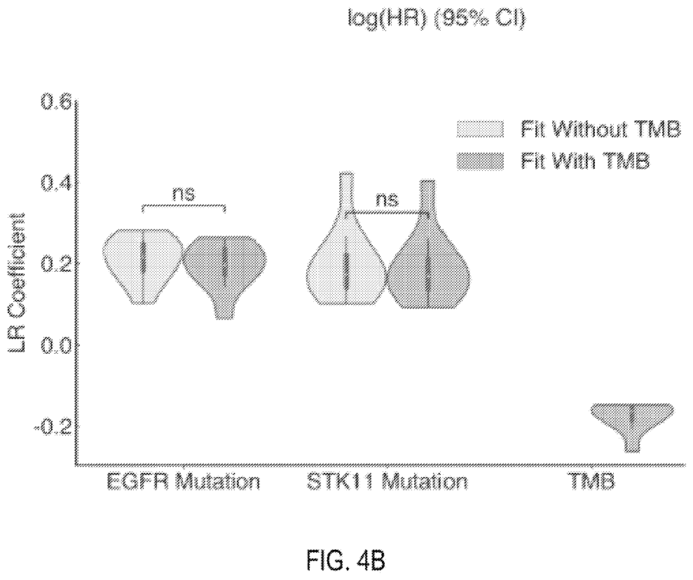

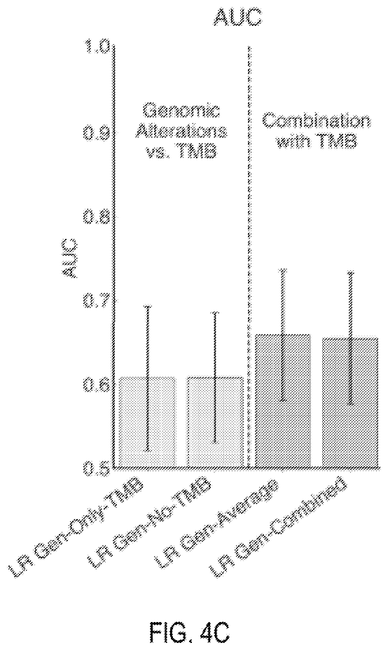

Features derived from clinical sequencing data from MSK IMPACT may be assessed. A 468-gene targeted next generation sequencing assay may be performed on FFPE tumor tissue along with matched normal specimens (i.e., blood) from each patient to detect somatic gene alterations with a broad panel. Using multivariate analysis on progression free survival in the multimodal cohort, alterations of EGFR (n=22/247, 8.9%; adjusted hazard ratio [aHR]=2.14, 95% CI 1.06-4.31, p=0.03), STK11 (n=44/247, 17.8%; aHR=2.53, 95% CI 1.71-3.74, p<0.005) and tumor mutation burden (TMB) (median 7 mt/mb, range 0-90; aHR=0.14, 95% CI 0.02-0.88, p=0.04) exhibited statistically significant aHR in a multivariable analysis of oncogenes (EGFR, ALK, ROS1, RET, MET and BRAF), tumor suppressor genes (STK11), transcription regulator (ARID1A), and TMB (FIG. 4A). Logistic regression was used to determine the association between TMB and response (AUC=0.61, 95% CI 0.52-0.70). The predictive ability of genomic alterations commonly studied in NSCLC excluding TMB (AUC=0.61, 95% CI 0.53-0.69) was inferior to the model trained using TMB and genomic alterations (AUC=0.65, 95% CI 0.60-0.80). However, the model performed similarly using the average of TMB and genomic alterations (AUC=0.65) (FIG. 4C). These features were independent predictors; EGFR and TMB were uncorrelated (r=−0.03, 95% CI −0.15-0.09) as well as STK11 and TMB (r=−0.01, 95% CI −0.14-0.11), and inclusion of TMB had no impact on the coefficients of EGFR and STK11 in the logistic regression fit (FIG. 4B). These results were broadly consistent with reports, establishing their suitability in this cohort for multimodal data integration.

### F. Multimodal Integration Using Deep Attention-Based Multiple Instance Learning Improves Prediction Over Unimodal Features and Clinical Biomarkers

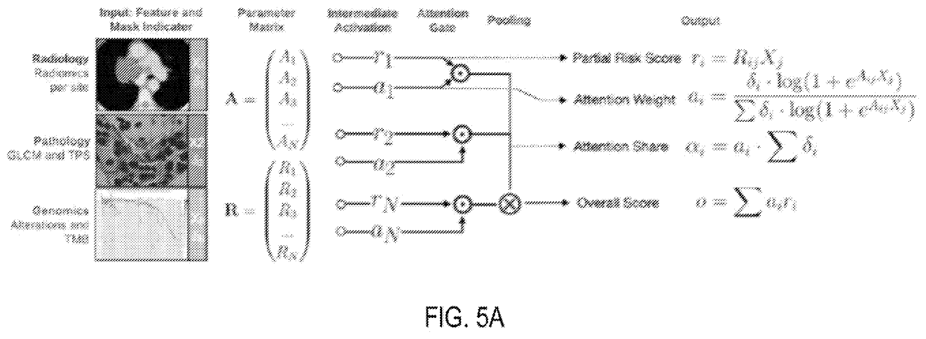

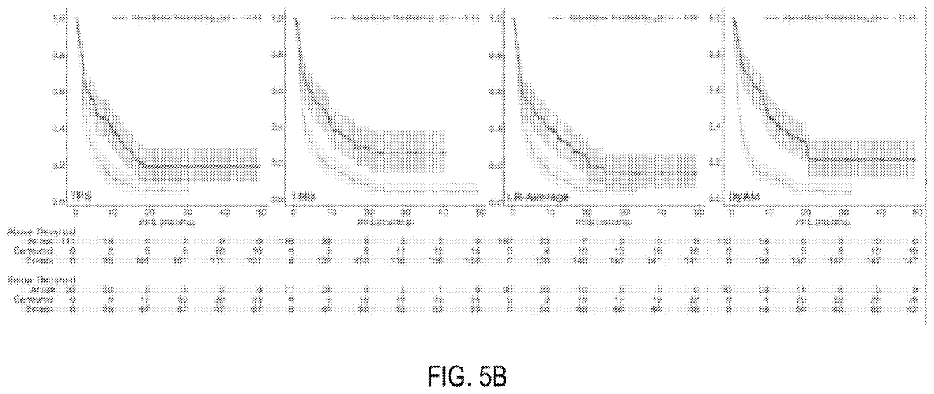

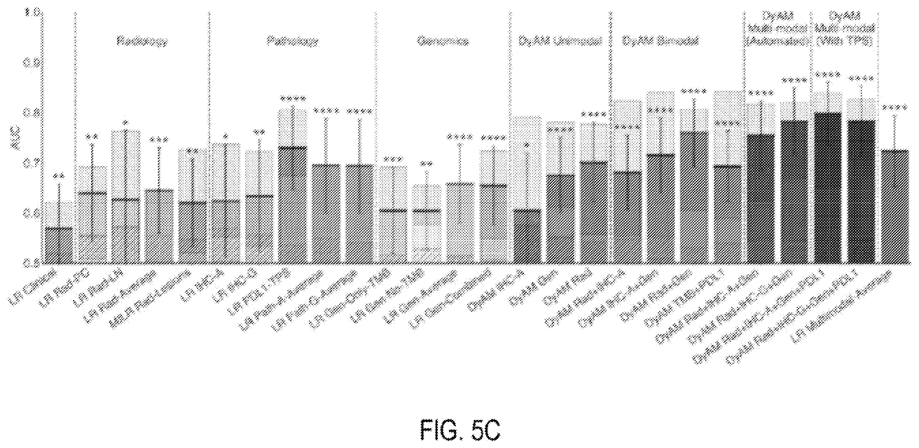

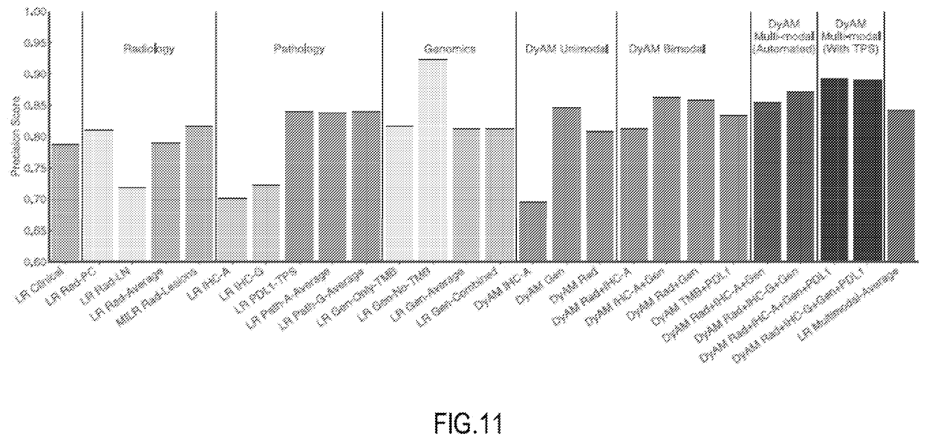

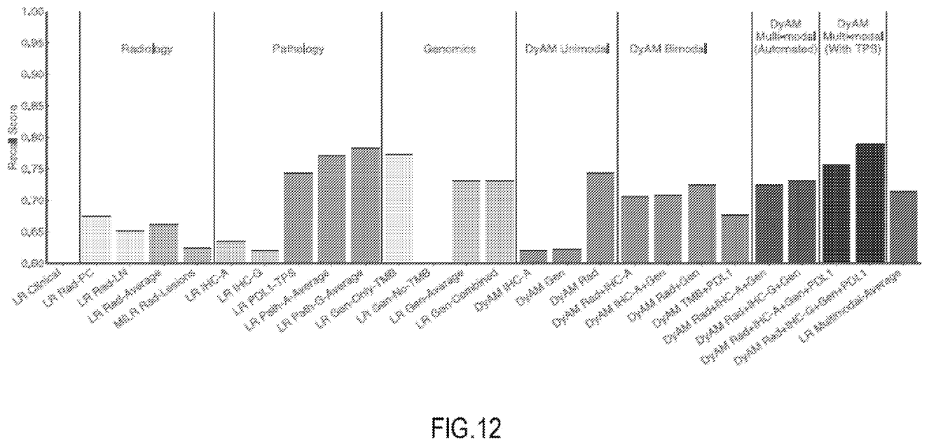

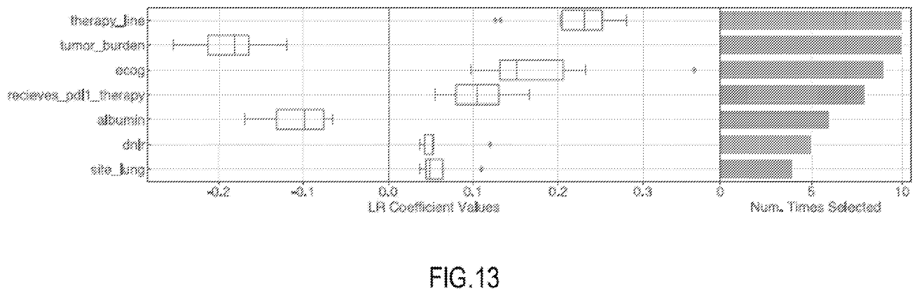

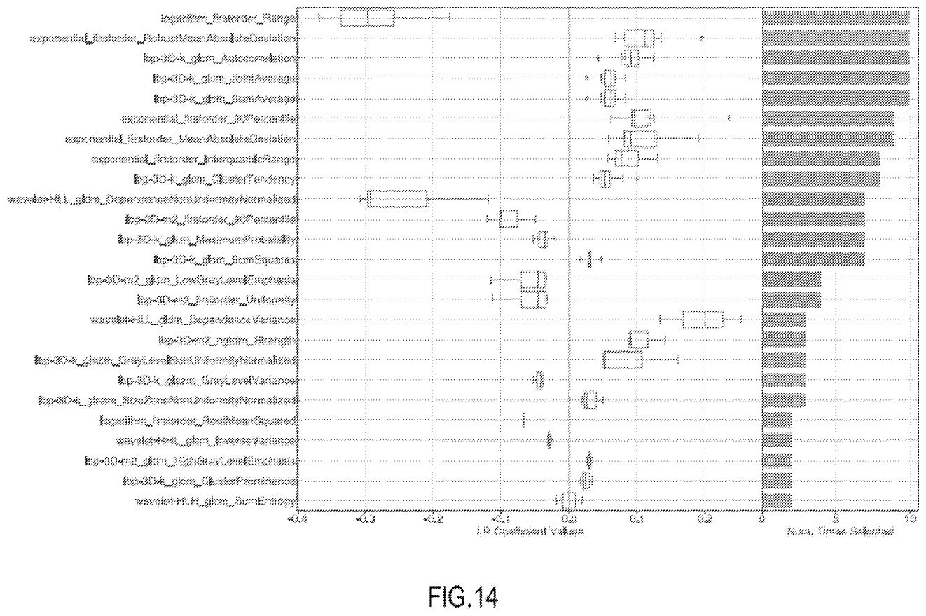

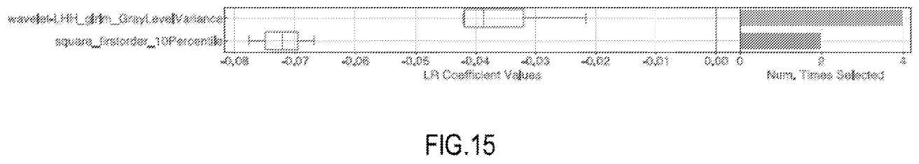

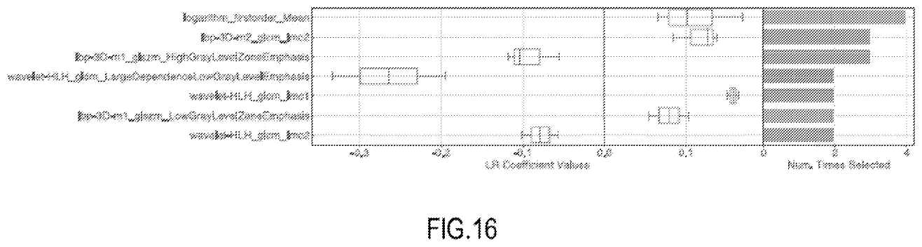

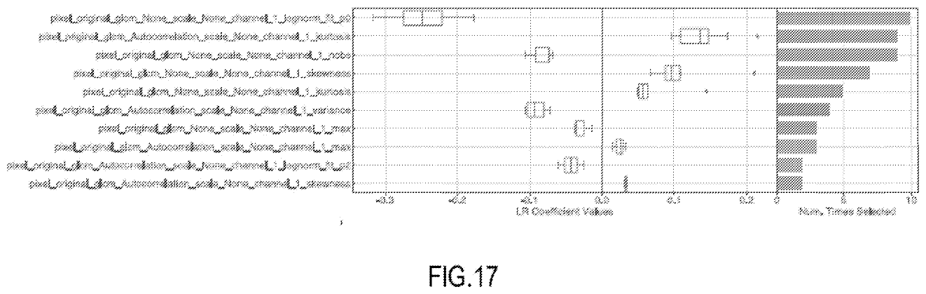

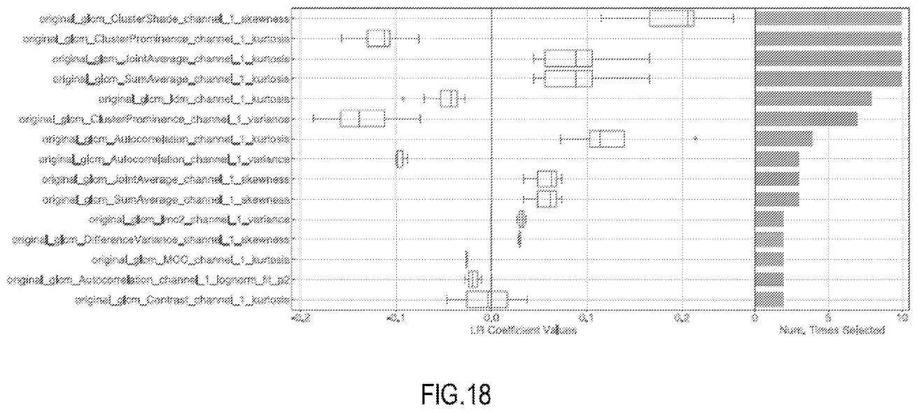

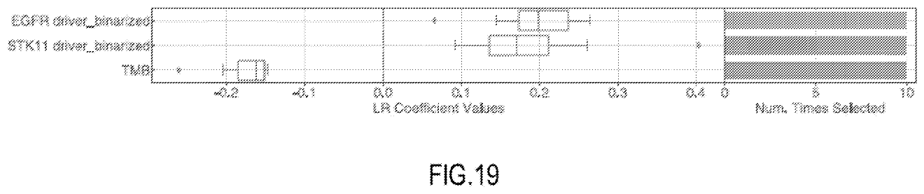

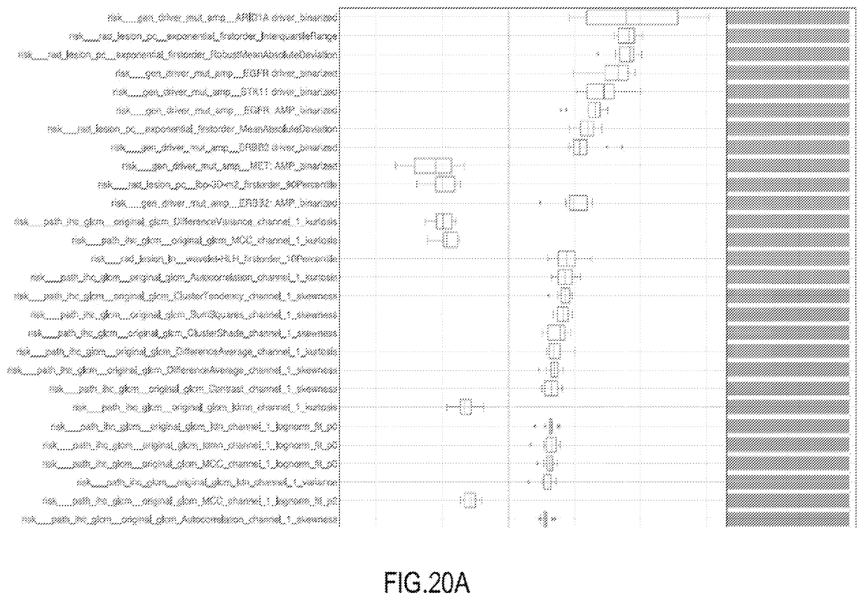

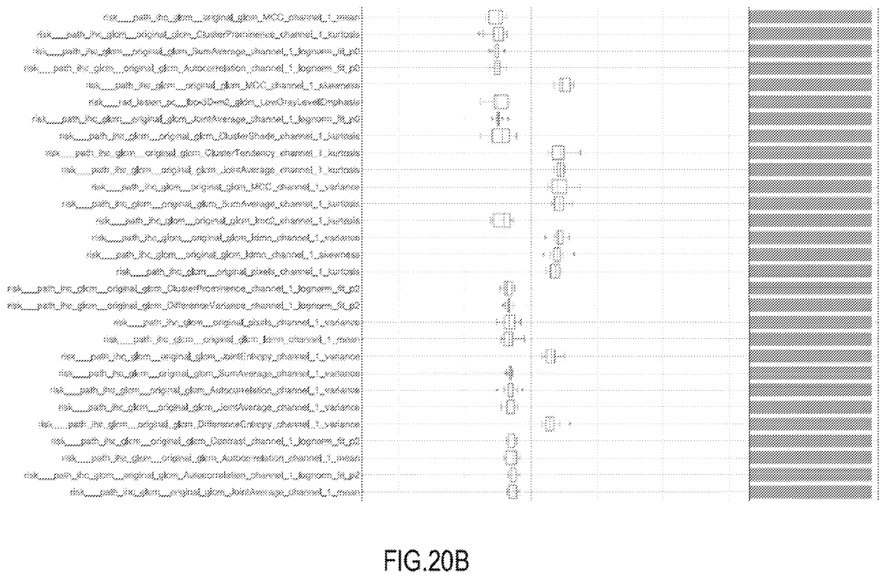

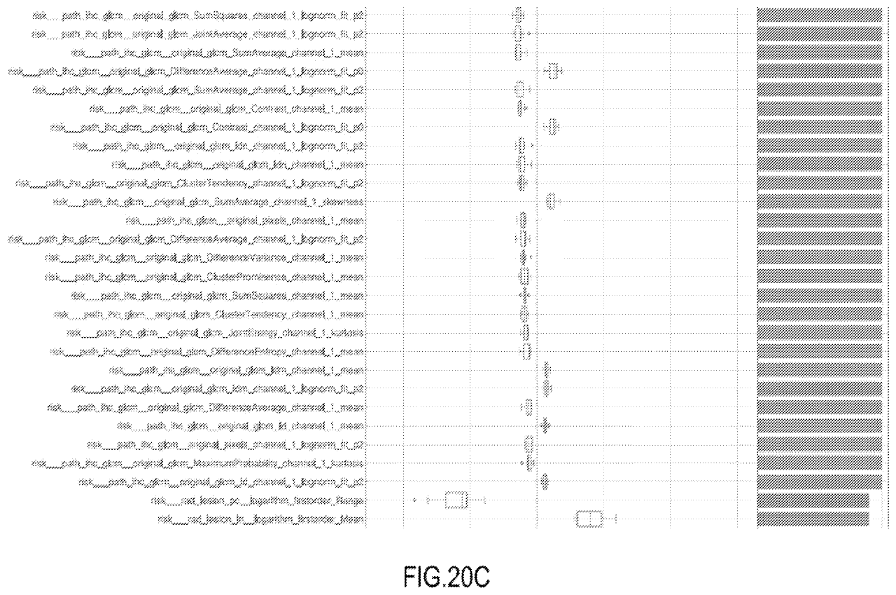

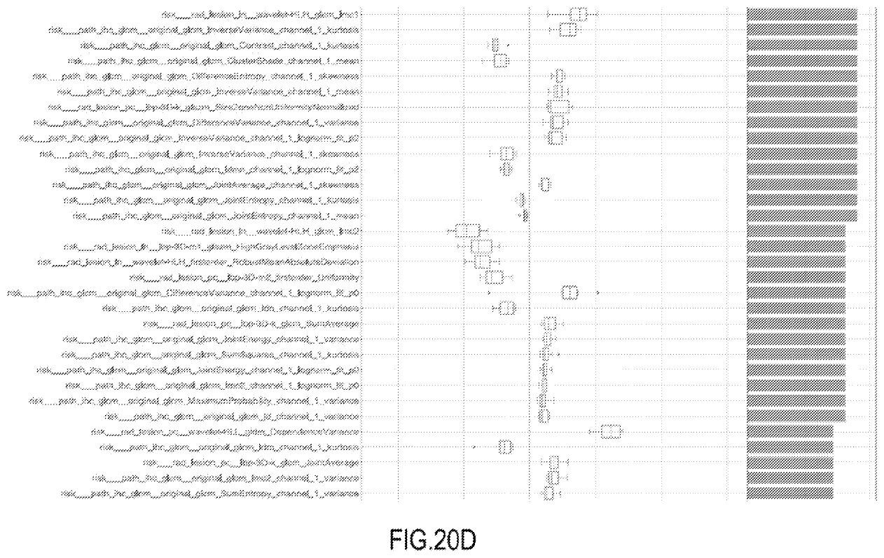

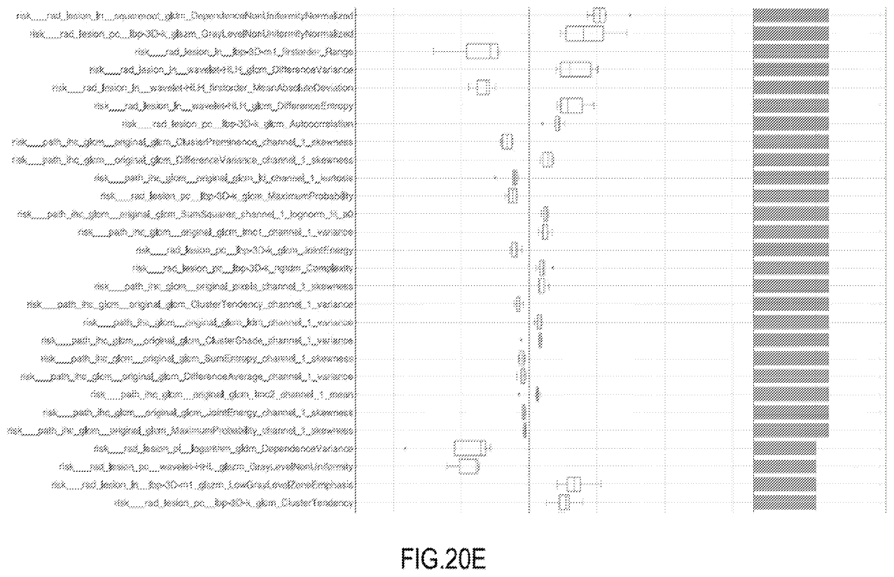

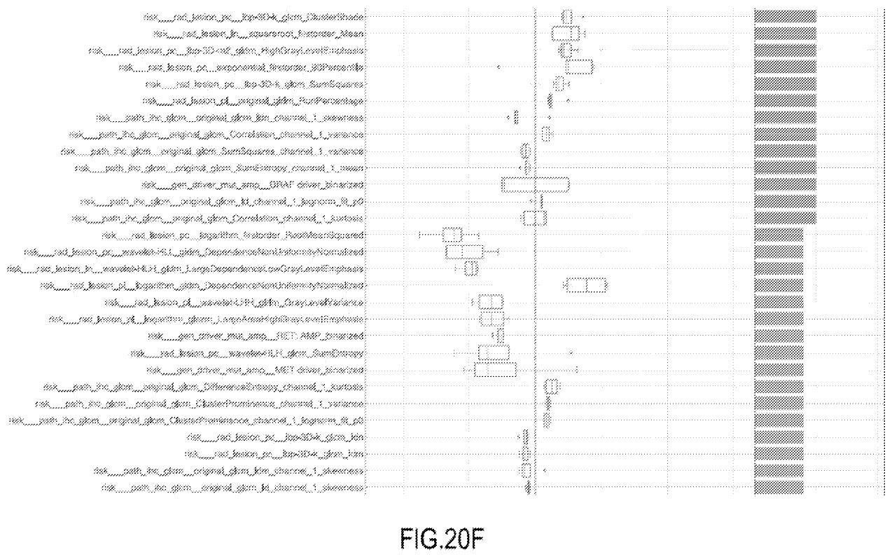

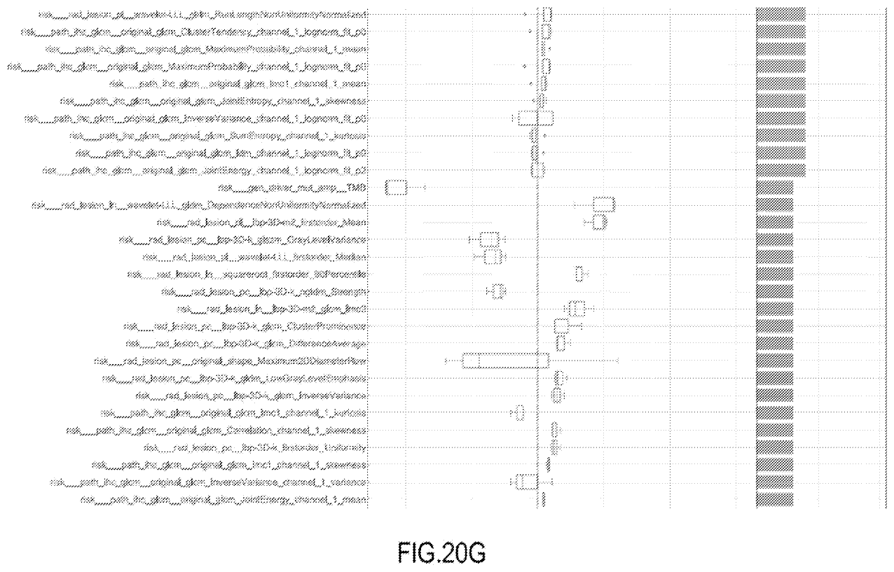

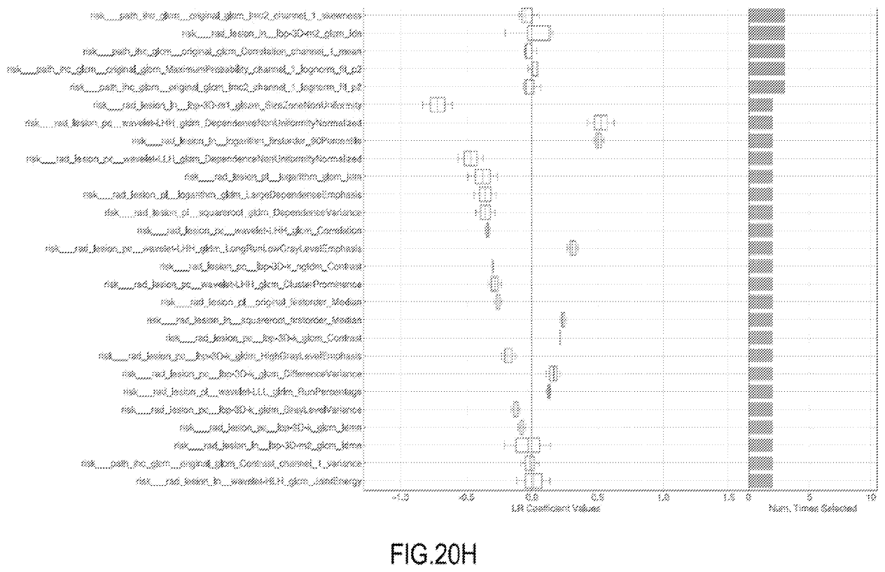

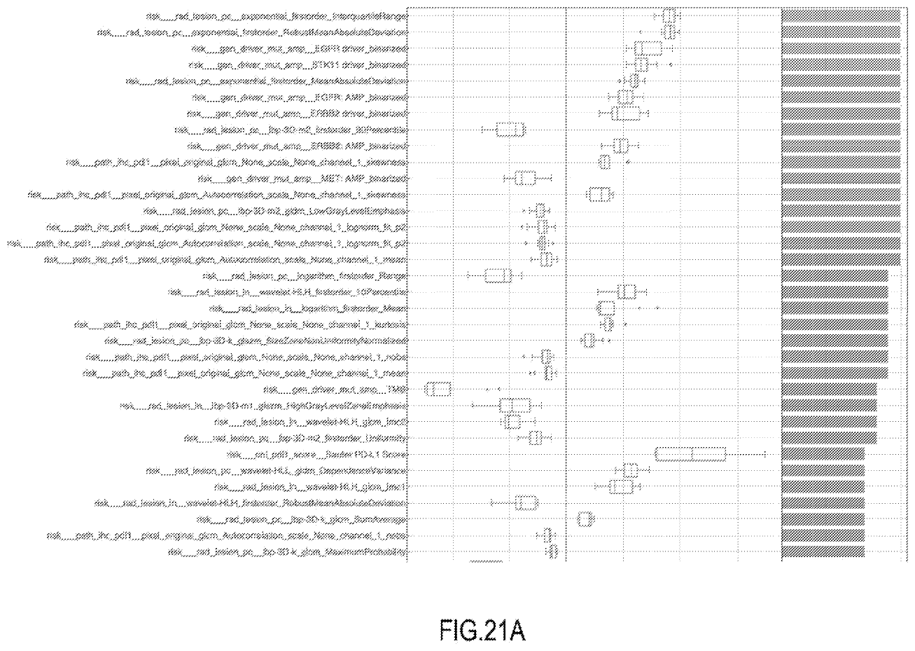

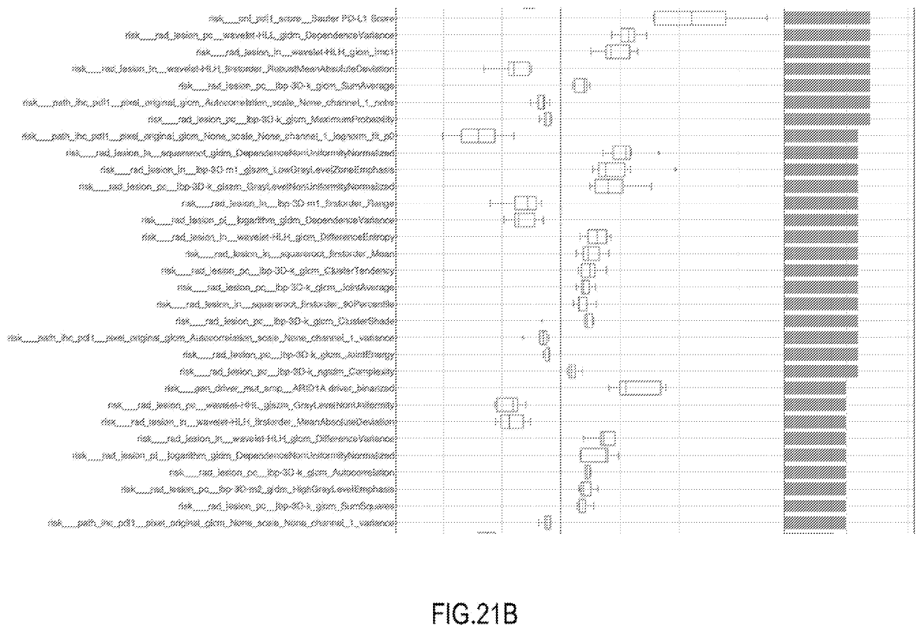

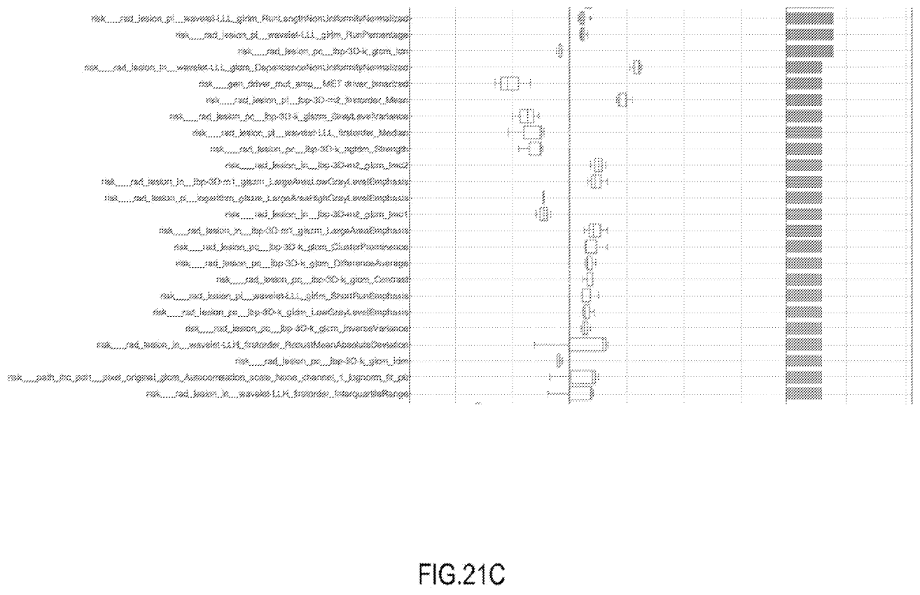

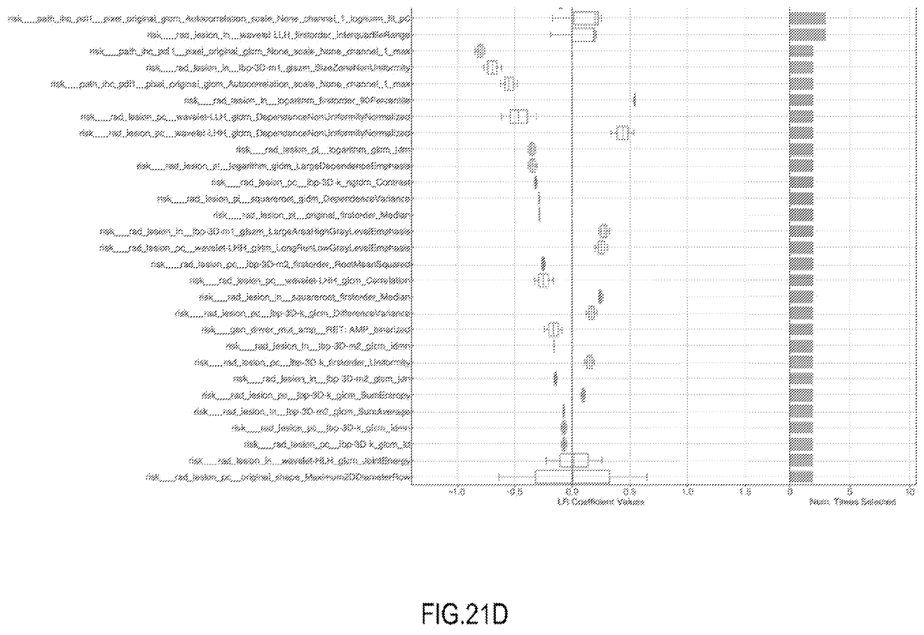

Having evaluated predictive capacity of unimodal features, a dynamic deep attention-based multiple instance learning model may then be implemented with masking (DyAM) to evaluate the impact of combining features from the complementary modalities of radiology, histology and genomics in predicting response to PD-(L)1 blockade (FIG. 5A). The DyAM model outputs include: risk attributed to each modality (partial risk score), attention the modality receives (attention weight and share), and the overall score and has the practical qualities of masking modalities in a given patient with no characterization, such as a tumor with negative PD-L1 expression or no segmentable disease in their CT scan. The performance of multimodal integration was assessed using Kaplan Meier analysis whereby stratification based on multimodal DyAM was more effective at separating high and low risk patients than the standard clinical biomarkers of TPS and TMB (FIG. 5B). Using this framework, unimodal features, and various combinations of bimodal and fully multimodal features may be systematically compared (FIG. 5C, F1, precision and recall scores shown in FIGS. 10-12, model coefficients are shown in FIGS. 13-21). In general, layering of complementary feature sets improved performance both within and between modalities. For example, DyAM integration of site-specific radiologic features improved prediction from AUC=0.65, 95% CI 0.57-0.73 to AUC=0.70, 95% CI 0.62-0.78. Furthermore, a bimodal DyAM model integrating radiological data and PD-L1 derived features (both TPS and IHC-texture) resulted in AUC=0.68, 95% CI 0.61-0.75, while the combination of PD-L1 derived features and genomic features resulted in AUC=0.72, 95% CI 0.65-0.79. Combining radiologic and genomic features resulted in the highest bi-modal performance (bimodal AUC=0.76, 95% CI 0.69-0.83). Each of these bimodal features improved on either unimodal feature set alone. The best performing, fully automated approach, using 3 modes of data included all GLCM features derived from digitized PD-L1 slides (IHC-G), with an AUC=0.78 (95% CI 0.72-0.85). Finally, using 3 modes of data with the pathologist derived TPS score resulted in the highest accuracy with AUC=0.80, 95% CI 0.74-0.86. This was in contrast with averaging the logistic regression scores from all modalities (AUC=0.72, 95% CI 0.65-0.79). All multimodal DyAM model results were significantly higher than null hypothesis AUCs obtained via permutation testing.

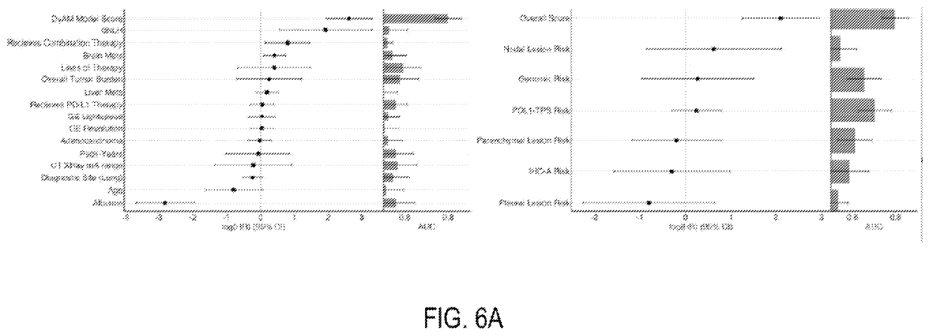

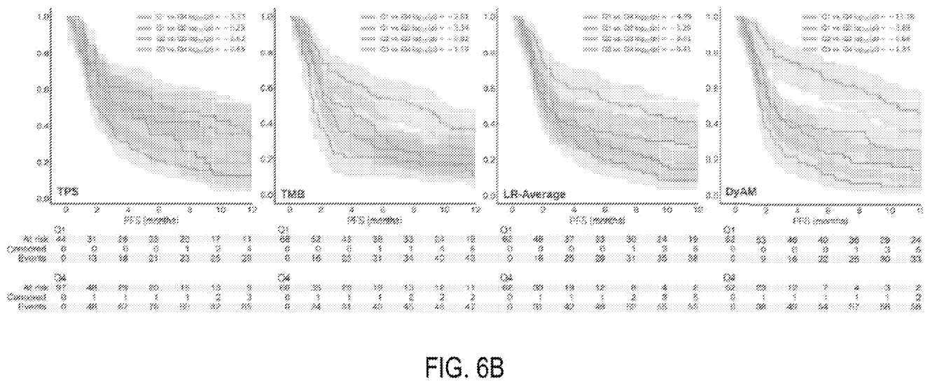

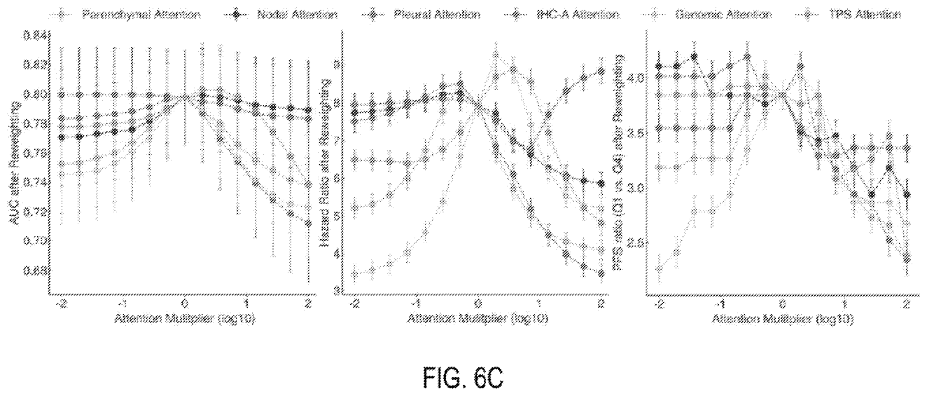

The DyAM model may be compared to established biomarkers of immunotherapy response as well as clinical confounders using multivariable Cox regression (FIG. 6A). The resulting overall score, DyAM-risk, was used as input to a multivariable Cox proportional hazards model with derived neutrophil to lymphocyte ratio (dNLR), pack-years smoking history, age, and albumin, tumor burden, presence of brain and liver metastases, tumor histology and scanner parameters. The resulting c-index was 0.74 with several significant features: dNLR (HR=6.87, 95% CI 1.76-26.77, p<0.005), DyAM-risk (HR=13.65, 95% CI 6.97-26.77, p<0.005), albumin (HR=0.06, 95% CI 0.02-0.14, p<0.005), brain metastasis (HR=1.51, 95% CI 1.09-2.09 p=0.01) and receiving combination therapy (HR=2.23 95% CI 1.16-4.29, p=0.02). When comparing the classifier performance against the logistic regression risk scores, only the integrated model was significant (FIG. 6A). The cohort may be divided into quartiles using the DyAM score and performed corresponding Kaplan-Meier analysis, focusing on progression-free survival in the first 12 months to highlight the potential of DyAM to separate response groups early after treatment. Progression at 4 months was 21% for the lowest (protective) quartile and 79% for highest (risk) quartile (FIG. 6B), compared to 30% and 60% for the averaging method. Finally, the effect of reweighting individual data modalities (Attention Analysis—Alpine Plots) on the overall model performance (FIG. 6C) may be assessed. In patient subsets with the data modality present, it is observed that the removal of lung parenchymal nodule CT texture and genomic alterations has the greatest effect on AUC while the model was robust to the removal of IHC-texture and PD-L1 TPS. Non-linear relationships between the data modalities indicate an effect of the weighting scheme used within DyAM. At four months the ratio of progression events between the lower and higher quartiles was 3.8 (95% CI 3.7-4.0), which decreases sharply when removing either the CT texture (decreasing to 3.2 (95% CI 3.1-3.3)) or genomic alterations (decreasing to 2.3 (95% CI 2.2-2.4)). However, this early separation did not manifest from either modality in isolation. Model performance decreases for all modes as unimodal attention increases, and the DyAM model outperformed simple averaging, highlighting the effect of the multimodal integration method.

### 3. Discussion

The integration of biomedical imaging, histopathology and genomic assays to guide oncologic decision-making is still in a preliminary phase. Herein, it is shown that machine learning approaches that automatically extract discriminative features from disparate modalities result in complementary and combinatorial power to identify high and low risk patients with NSCLC who received immunotherapy. This represents a proof-of-principle that information content present in routine diagnostic data including baseline CT scans, histopathology slides, and clinical next generation sequencing can be combined to improve prognostication for response to PD-(L)1 blockade over any one modality alone and over standard clinical approaches. Integration of these data presents technical difficulty and infrastructure cost. However, the results indicate the potential of integrative approaches. To enable growing interest in deploying data infrastructure to automate the collection, organization, and featurization of the data included in this study, the workflows and software are provided for use by the broader community in other cohorts, and can be applied beyond NSCLC to other cancers and diseases.

To enable the study, domain-specific experts were consulted to curate features in our dataset. Curation involved segmentation of malignant lesions within CT scans by thoracic radiologists (such as those shown in FIG. 2B) and annotation of digitized PD-L1 IHC slides (such as those used train the machine learning classifier to compute the tumor segmentation mask in FIG. 3A) and adjudication of PD-L1 expression by a thoracic pathologist. Genomic and clinical features were limited to those with known associations to NSCLC and immunotherapy outcomes. Heterogeneity of the disparate data modalities presented a unique challenge in their integration. For instance, not all patients presented with segmentable disease in radiological CT images. In patients with segmentable disease, there were multiple lesions across disparate sites, which presented the challenge of developing a whole-patient characterization. A separate challenge was present when characterizing PD-L1 expression patterns, which are not defined for PD-L1-negative tumors. Finally, the most optimal combination of these features is not known, and post-fit linear combination or averaging techniques could ignore important interactions and correlations between these modalities. The attention-gate of DyAM allows for non-linear behavior across the input modalities. The use of attention gating and the generation of partial risk scores has added benefit; it allows for higher-level analysis of multi-modal data, such as automatically identifying regions of feature space where certain modalities are more or less predictive. The result was an interpretable, data-driven multimodal prediction model which was also robust to missing data. Reassuringly, the multi-modal DyAM model was not only able to predict short term objective responses better than any modality separately or linearly combined, but also led to enhanced separation of the Kaplan-Meier survival curves that reflected discriminative power within the first few treatments. This is further evidence that the model could achieve early stratification of true responders from non-responders, an important criteria for predictive biomarkers and future clinical management decisions. Furthermore, the attention analysis of the DyAM model revealed that all data modalities (radiomics, genomics, and pathology) are drivers of this early stratification.

A limitation of the analysis was the size of the multimodal cohort assembled and the restriction to a single center. In order to ensure consistent training data quality, only CT scans that were performed within one institution were included. Inclusion of CT scans from external institutes warrants further study to investigate the effect of various machine acquisition parameters on model sensitivity. Each scan was reviewed by radiologists who routinely perform RECIST reads for clinical trials. Digitized PD-L1 IHC slides were similarly chosen from a single center given differences in staining quality in PD-L1 IHC among different laboratories and the use of several different antibodies in clinical practice among institutions. Similarly, existing commercial and institution-specific targeted next generation sequencing panels differ in breadth of coverage and germline filtering techniques, which can introduce challenges for data integration, and not all institutions sequence patient matched normal specimens to identify germline mutations. These challenges can be mitigated by training models with data from multiple sites to either predict clinical outcomes directly or to perform segmentation in pathological or radiological imaging for downstream analysis. However, a multisite training strategy requires comparable dataset sizes across sites with consistent and rigorous annotations in order to properly normalize models for heterogeneity and extract robust features. It therefore remains an open question as to how these models would generalize across technical platforms or institutional sources of variation. Federated learning may provide a principled solution to this challenge, however its practical use is at very early stages of adoption. Although an external validation cohort was unable to be obtained given the complexity of the data modalities, internal single modality validation cohorts for CT scans and histopathology slides were used as full hold-out sets to validate the findings from the multimodal cohort. Indeed, the models with significant and robust performance in the multimodal cohort showed stable performance in the radiology and pathology cohorts. Despite best efforts, underperforming models encounter statistical limitations that can be best minimized with further data collection.

Another constraint of the analysis was the use of RECIST derived response endpoints. RECIST outcomes were chosen instead of directly predicting survival metrics to minimize effects from confounders such as indolent disease, future lines of therapy and death unrelated to NSCLC. However, RECIST responses are characterized from CT, which does not take into account possible histological or genomics changes in the tumor. Additionally, while correlation of RECIST to survival has been observed in NSCLC, other pancancer studies have found response endpoints to be an unreliable surrogate. Future studies are required to investigate the utility of RECIST endpoints and potential alternatives.

Scaling and extending the model to incorporate external data would require annotation algorithms to segment CT scan lesions or distinguish tumor from normal tissue in PD-L1 IHC slides to reduce expert burden. Alternatively, large de-identified datasets from many sites may overcome the need for manual annotation by developing reliable deep learning models on unannotated data, which can be directly included as part of the DyAM model. In the future, assembly of large well-annotated multi-institutional training datasets may lead to development of robust multimodal classifiers that serve as powerful biomarkers. These decision aids could be integrated into routine clinical care and used to quickly and precisely distinguish responders and non-responders to treatment.

Along with the integration of multiple sites, a deeper understanding of features extracted from the data modalities and their relationships to known functional cancer pathways could also aid in feature selection. For example, radiomics characterizations involve the extraction of thousands of features which can be used together to broadly encapsulate intatumoral heterogeneity, but there have been few studies using correlative molecular data to infer functional relationships. This task is further complicated by the fact that many radiomics features are correlated to each other. One study used gene set expression analysis and found an association between radiomics and cell cycle progression and mitosis. Similarly, correlative molecular data could aid in a more principled selection of features which comprise the PD-L1 IHC texture characterization.

These results reaffirm the principle that existing data from multiple cancer diagnostic modalities can be annotated, abstracted, and combined using computational and machine learning methods for next generation biomarker development in NSCLC immunotherapy response prediction. The resulting DyAM model is a promising new approach to integrate multimodal data, and future models using larger datasets may make it possible to augment precision oncology practices in treatment decision making.

### 4. Methods

### A. Data Infrastructure to Support Multimodal Data Integration

The computational and data infrastructure to support the ingestion, integration, and analysis of the multimodal dataset was built through the MSK MIND (Multimodal Integration of Data) initiative. Data pipelines were built to extract and de-identify clinical, radiology, pathology, and genomics data from institutional databases. A data lake was built to ingest and manage all data with an on-premise cluster. Workflows were implemented to source the data lake to facilitate analyses using radiology and pathology annotations. All data, metadata, and annotation described below were integrated for multimodal analysis.

### B. Clinical Cohorts

Following approval of the institutional review board, the multimodal cohort was formed using the following inclusion criteria: patients with stage IV NSCLC who initiated treatment with anti-PD-(L)1 blockade therapy between 2014-2019 at the study institution who had a baseline CT scan, baseline PD-L1 IHC assessment and next generation sequencing by MSK-IMPACT. Patients who received chemotherapy concurrently with immunotherapy were not included. 247 patients met inclusion criteria for the training cohort.

The radiology (n=50) validation cohort included patients with a baseline CT which included the chest (+/−abdomen/pelvis) containing lung lesions>1 cm. The pathology (n=52) validation cohort included patients with a biopsy showing PD-L1-positive (TPS≥1%) NSCLC that was digitized at MSK. Baseline characteristics of the multimodal, radiology and pathology cohorts are shown in Table 1. Best overall response was assessed via RECIST v1.1 by thoracic radiologists trained in RECIST assessment. Patients who did not progress were censored at the date of last follow up. Progression free survival (PFS) was determined from the date of initiating PD-(L)1 blockade therapy until the date of progression or death. Overall survival (OS) was determined from the date of initiating PD-(L)1 blockade therapy until the date of death. Those who were still alive were censored at their last date of contact. Clinical, radiologic, pathologic, and genomic data was housed in a secure Redcap database.

### C. Computerized Tomography (CT) Scans

The baseline CT scan was defined as the closest contrasted scan including the chest performed within 30 days of starting PD-(L)1 blockade therapy at MSK. Scans were anonymized and quality control was performed to ensure de-identification. Scans were separated into the DICOM format and metadata. All patients underwent multisection CT performed as part of standard clinical care for clinical staging of pulmonary malignancy. CT studies were all performed at the institution (Lightspeed VCT, Discovery CT 750HD; GE Healthcare) and were submitted and uploaded to the picture archiving and communication system.

### D. Radiological Segmentation

The study was limited to chest imaging to ensure homogeneity of the imaging protocol used. As a result, chest lesions were considered. Lesion segmentation of primary lung cancers and thoracic metastases were performed manually by three radiologists (NH and AA with eight years of post-fellowship experience, AP with one year of post-fellowship experience). Each lesion was segmented by a single radiologist, reviewed by a second and disagreements were resolved with a third. While all radiologists were aware that the patients had lung cancer, they were blinded to patients' prior treatments and outcomes.

Target lesions were selected in accordance with RECIST v1.1 criteria (maximum of 5 target lesions and up to 2 target lesions per organ). Lesions that were segmented included lung parenchymal, pleural, and pathologically enlarged thoracic lymph nodes. Lung and pleural lesions were included when measured as >1.0 cm in the long axis dimension and lymph nodes when>1.5 cm in the short axis dimension.

Segmentations were performed on contrast enhanced CTs with 5 mm slices and soft tissue algorithm reconstructions. The segmenting radiologist had access to the clinical text report and PET scan images during segmentation as guides. Lung and soft tissue windows (window level: −600 HU and width: 1500 HU, and window level: 50 HU and width: 350 HU, respectively) were used when appropriate to visually delineate volumes of interest (VOI) from lung tissue, large vessels, bronchi, and atelectasis. Cavitary lesions, lung lesions indistinguishable from surrounding atelectasis, and streak artifacts were excluded. Segmented target lesions were categorized and labeled separately by location for textural feature analysis.

### E. Radiomics Feature Analysis

Three thoracic radiologists (NH, AP and AA) used dictated radiology text reports, PET scan images and RECIST criteria to guide segmentation. Areas of ambiguity, such as image artifacts from surgical staples, were excluded. A total of 337 lesions from 187 patients, classified into lung parenchymal, pleural and lymph nodes were segmented. The predictive capacity of features extracted from lesions segmented CT scans were analyzed. A variety of radiomics features were computed using all filters available in pyradiomics, resulting in 1,688 features. To ease the training of the predictive model, the number of features were reduced by requiring stability with respect to small perturbations of the original segmentation using the method to assess robustness of radiomics features. In this method, the original segmentation is perturbed 10 times, then radiomic features are computed from each perturbation. A robustness z-score may be defined as the ratio of the average inter-lesion variance across the 10 perturbations and the feature intra-lesion variance average across the entire multimodal cohort. This value ranges from 0-1, and only features with z-scores less than 0.15 were considered. This ensured that, on average, selected features only vary slightly (˜15%) across the perturbations with respect to its total dynamic range. The same procedure was implemented in the analysis of the radiology cohort.

### F. PD-L1 Immunohistochemistry

IHC was performed on 4 m FFPE tumor tissue sections using a standard PD-L1 antibody validated in the clinical laboratory at the study institution. Staining was performed using an automated immunostaining platform using heat-based antigen retrieval employing a high pH buffer for 30 min. A polymeric secondary kit was used for detection of the primary antibody. Placental tissue served as positive control tissue. Interpretation was performed on all cases by a thoracic pathologist (JLS) trained in the assessment of PD-L1 IHC. Positive staining for PD-L1 in tumor cells was defined as the percent of partial or complete membranous staining among viable tumor cells, known as the tumor proportion score (TPS). A negative score was defined as staining in <1% of tumor cells or the absence of staining in tumor cells. Slides that did not meet the minimum number of tumor cells for PD-L1 TPS assessment (i.e., <100 tumor cells) were not included. The same procedure was implemented to characterize the pathology cohort.

### G. PD-L1 Tissue Analysis

PD-L1 IHC-stained diagnostic slides were digitally scanned at a minimum of 20× magnification for 201 patients using an Aperio Leica Biosystems GT450 v1.0.0. A deep learning classifier implemented in the HALO AI software was trained to recognize areas of tumor in PD-L1-stained tissue. The training involved annotations across multiple tissue slides to subsequently train the DenseNet AI V2 classifier. The following annotation classes were included: tumor, stroma, lymphocytes, necrosis, fibroelastic scar, muscle, benign lung tissue and glass (absence of tissue). Multiple slides were used to train the classifier to account for site heterogeneity. The trained classifier was then employed across all PD-L1 IHC slides available for the multimodal cohort. Each slide was subsequently manually assessed for tumor segmentation by a thoracic pathologist (JLS) and assigned a specificity score. This score was defined as the proportion of tissue being identified as tumor being correct. Slides with scores below 95% were then manually annotated.

### H. PD-L1 Staining Pattern Quantification

Once analyzed in the HALO AI software, tumor masks were exported and applied to the original PD-L1 tissue image. The masked tissue image was de-convoluted to separate the PD-L1 IHC from the hematoxylin blue counterstain. Gray-level co-occurrence matrices (GLCMs) were used to characterize PD-L1 expression. GLCMs are commonly used in image processing to quantify the similarity of neighboring pixels. Pixel-wise GLCM features were extracted using the pyradiomics package. This is performed within pyradiomics by computing GLCM features within a 3×3 kernel around each pixel in the image. Summary statistics including mean, standard deviation and autocorrelation were computed from the pixel-wise GLCM map for use in downstream analysis.

### I. Genomics Analysis

All patients had panel next generation sequencing performed on their tumor prior to the start of treatment. The platform used was the FDA-authorized MSK-IMPACT platform, which includes somatic mutations, copy number alterations and fusions in 341-468 genes most commonly associated with cancer. Genomic alterations in genes commonly associated with non-small cell lung cancer (NSCLC) and thought to associate with anti-PD-L1 therapy response were extracted from cBioPortal. This included altered oncogenes (EGFR, ALK, ROS1, RET, HER2/ERBB2, BRAF, MET), altered tumor suppressors (STK11) and altered transcription regulators (ARID1A). All alterations were annotated by OncoKB to determine whether they are potentially oncogenic or driver events. TMB, a measure of the number of somatic mutations per megabase of the interrogated genome, is an FDA-approved predictive biomarker of immunotherapy response in NSCLC. TMB was calculated based on all identified mutations divided by the coding region captured in the MSK-IMPACT panels. Multivariable analysis was conducted using Cox-proportional hazards modeling adjusting for the genes described as well as TMB.

### J. Logistic Regression (LR)

Logistic regression with elastienet penalty was used to predict binary outcomes given feature vectors. The implementation using the saga optimizer, C=0.1, a 11-ratio of 0.5, and balanced class weights were used.

### K. Multiple Instance Logistic Regression (MILR)

Multiple instance learning is a class of machine learning where sets of training data (bags) share a common label. Attention-based pooling extended the multiple instance learning paradigm to assign attention-based weights to each instance, which are a function of the instance features and optimized for prediction of an outcome. This technique is designed for an unfixed number of input instances with fixed feature size, such as the 1-5 texture feature vectors for each lesion encountered in the radiology analysis. The final output score is a weighted sum of the same logistic regression model applied for each lesion, where the weights are dynamically determined. In the single-lesion case, similar results were able to be recovered as compared with the standard logistic regression. This technique treats each instance equally as there is a single set of parameters shared across all instances.

The model was developed and fit using pytorch 1.8.0, with a hidden size of 32, balanced class weights, binary cross-entropy loss, 0.005 learning rate, 250 steps and L2 regularization of 0.005 using the Adam optimizer.

L. Multi-Modal Dynamic Attention with Masking (DyAM)

Multiple instance learning typically involves a single group, or bag, of homogenous instances. However, in the multimodal case heterogenous instances may be encountered. Furthermore, even though the textures derived from each lesion are the same shape, lesions in different sites may map to the predictor in different ways, such that a shared set of parameters may not be optimal. A MIL paradigm may thus be encountered where there is a fixed-sized bag of heterogeneous instances, or modes. For this study, the modes of data utilized were 1D feature vectors derived from: PD-L1 IHC performed on diagnostic tissue, segmented radiological CT scans delineated by lesion site, and genomic alterations. The same dynamic pooling scheme may be used in the model. Each input mode has its own set of trainable parameters: one to map the input vector to the label (risk parameters), and one to map the input vector to an attention score (attention parameters), which competes with the other input modes in a similar way to the standard multiple instance learning model. Since attention-based weights are trainable, the model learns which input modes are most relevant to treatment response. In contrast to MILR, the final output score is a weighted sum of individually optimized logistic regression models applied for each modality; this technique treats each mode specifically, while still optimizing all parameters in concert. Finally, since zero attention corresponds to a modality having no correspondence to the predictor, this model was expanded by adding a masking function which sets any missing mode's attention to zero to address missing data (such as non-segmentable disease in CT). This may be called a Dynamic Attention with Masking model (DyAM).

The model was developed and fit using pytorch 1.8.0, with balanced class weights, binary cross-entropy loss, 0.01 learning rate, 125 steps and L2 regularization strength of 0.001 using the Adam optimizer. When only one input modality was present, the attention gate was disabled to reduce complexity.

### M. Attention Analysis

Given the normalized attention weights and risk scores per modality, one can reweight the attention layer to assess how an increase or decrease in a modality's attention impacts the model's performance. This can be done schematically by multiplying the attention vector ā by a multiple of the k-th unit vector mêₖ, taking the dot product with the risk vector r̅, and re-normalizing the score.

$$score = ( m {ê}_{k} ∘ \overline{a} ) \cdot \overline{r} / \sum ( m {ê}_{k} ∘ \overline{a} )$$

The new weights can be determined via:

$${a}_{i = k} = m {a}_{k} / ( 1 + ( m - 1 ) {a}_{k} ) , {a}_{i \neq k} = {a}_{i} / ( 1 + ( m - 1 ) {a}_{k} )$$

If aₖ=0.5 and m=10, the new attention of the k-th modality increases to 0.91, and the other modalities' attention weights decrease by a factor of 0.18. Conversely, at m=0.01, the attention of the k-th modality decreases to 0.01, and the other modalities' attention weights increase by a factor of 2. At high multiples, the model approaches a unimodal model dominated by that modality. At low multiples, the model approaches a N−1 model with that modality dropped out. m in the log₁₀ scale from −2 to 2 were scanned, and performance metrics (AUC, hazard ratio, PFS ratio) were recomputed at each step.

### N. Statistical Analysis

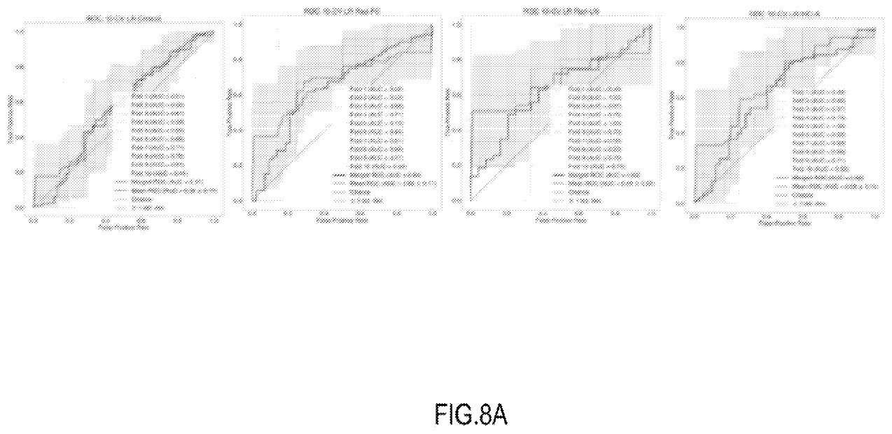

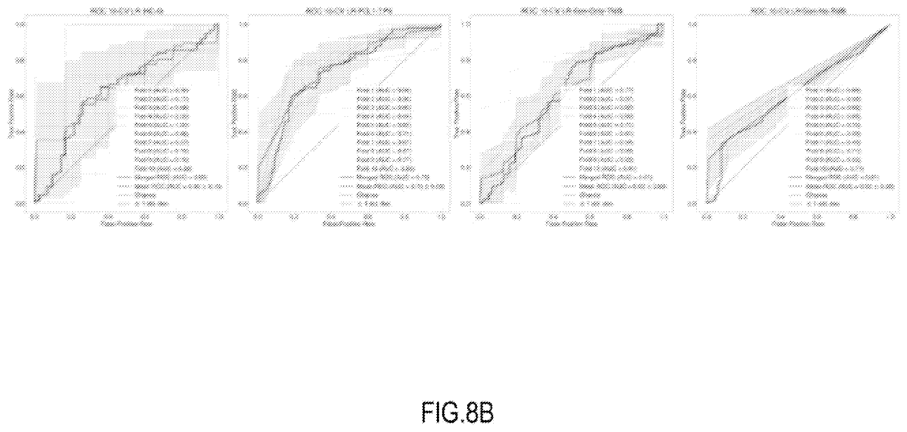

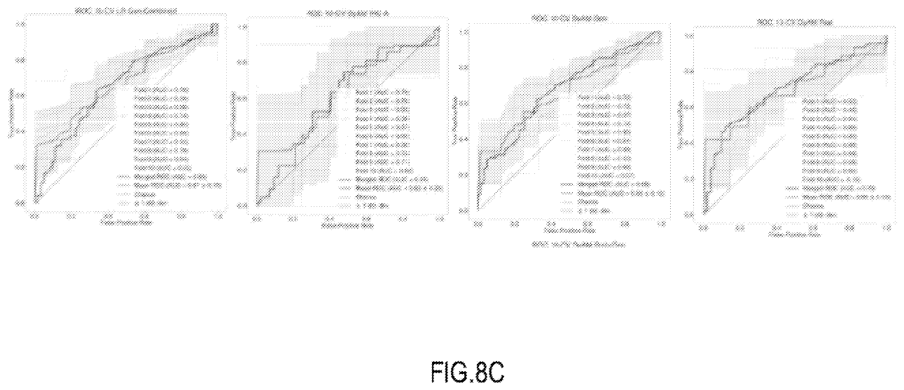

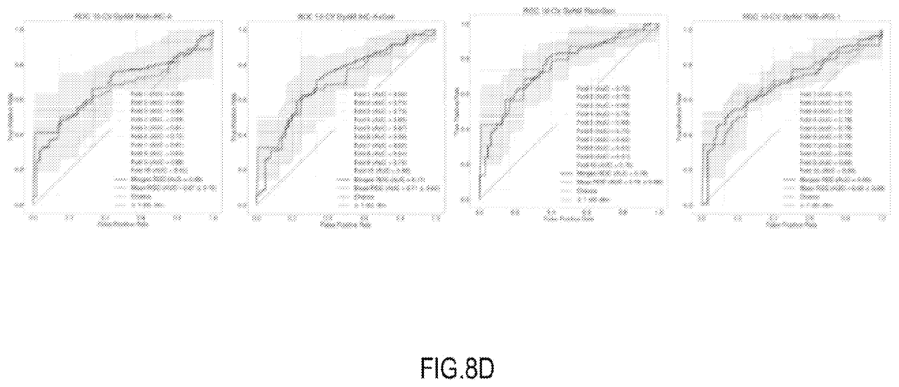

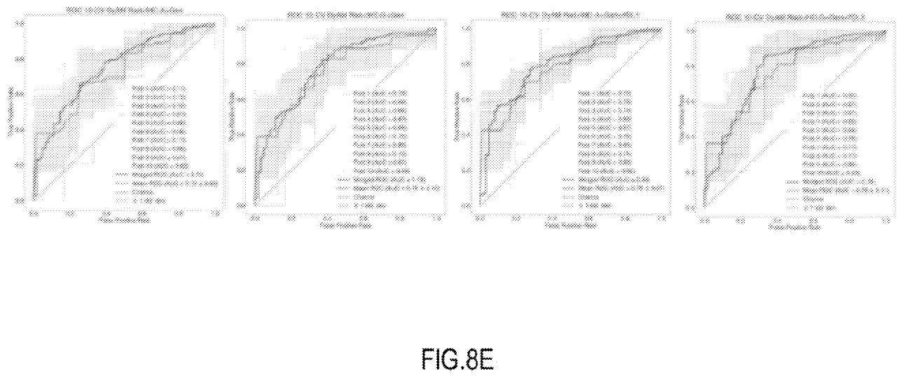

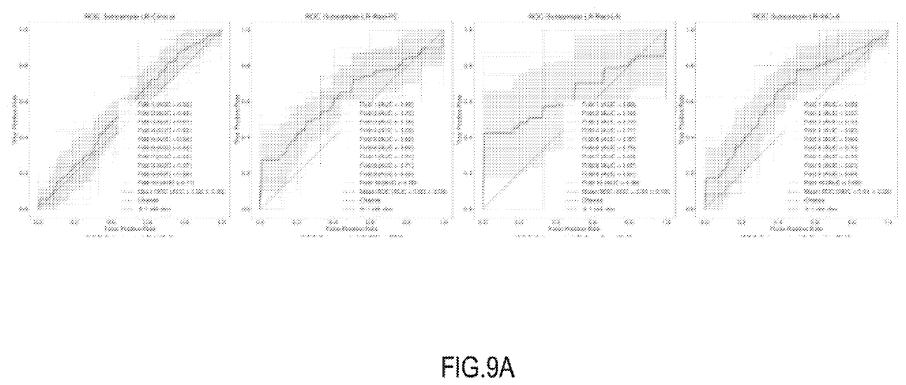

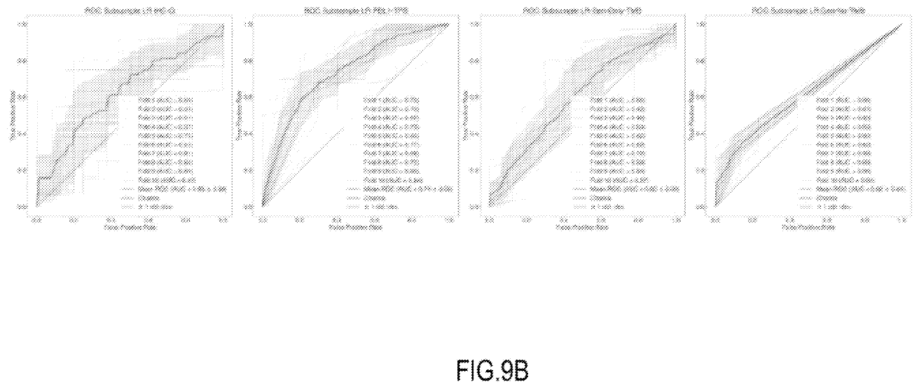

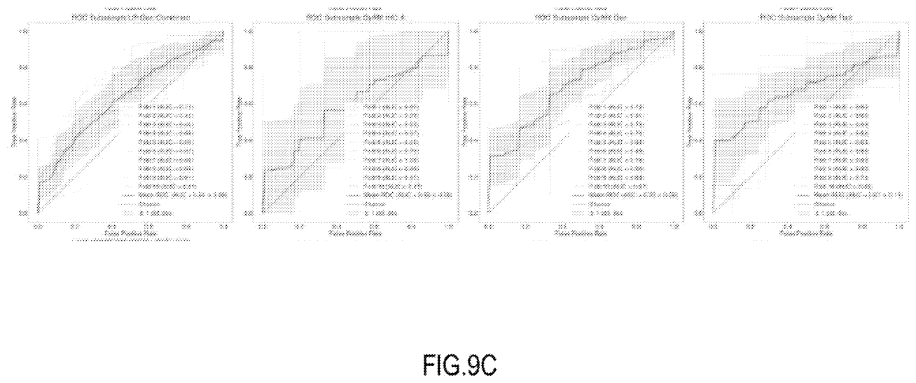

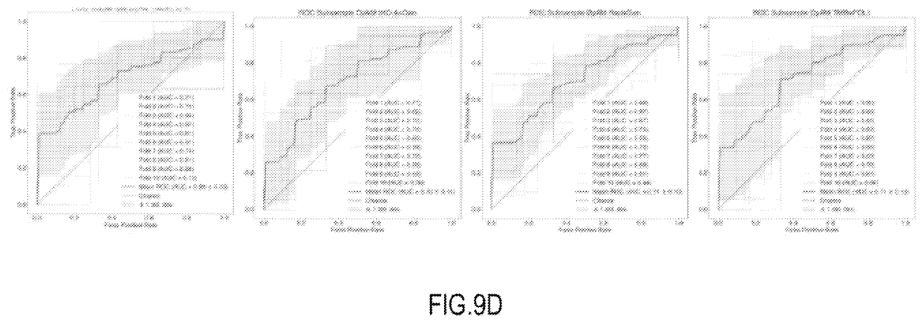

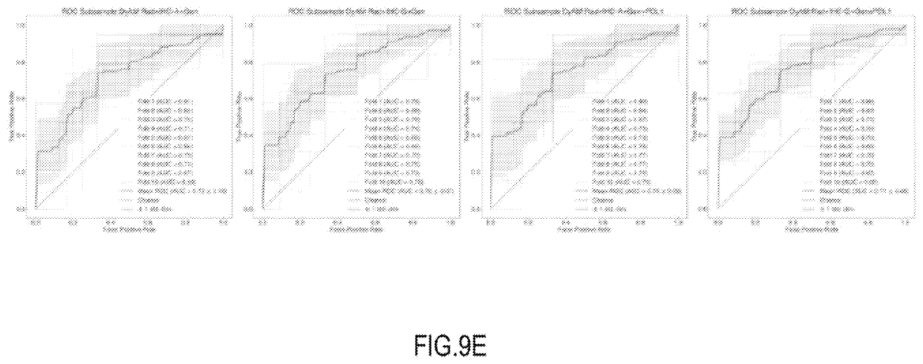

There were no formal sample size calculations performed in advance. The multimodal cohort was selected based on the existing number of patients at MSK who met all inclusion criteria. Area under the receiver operating characteristic curve (AUC) was used as a measure of performance of a biomarker on predicting response vs non-response. A 10-fold cross-validation was performed where scores from the validation folds were combined to form predictions across the entire multimodal cohort. All feature selection and feature rescaling was performed on the training fold in each iteration of the cross-validation analysis. The binary cut-off used to determine the predicted response was determined for each patient using only training data. Hyperparameters were established from the first fold and applied consistently for each model. To explicitly evaluate the sensitivity of model fitting to the data used for training, a subsampling analysis of model fitting may be performed to the data used for training. A subsampling analysis may also be performed where the data was sampled 100 times using 90% for training and the remaining 10% for testing. All models were trained in an end-to-end manner corresponding cross-validation across folds (FIG. 8) and subsampling (FIG. 9). The F1-score, precision and recall for all models may be seen in Table 1 and FIGS. 10-12. Model coefficients are shown in FIGS. 13-21. A multivariable cox proportional hazards model was developed with the Lifelines python package to compare the performance of this model to existing biomarkers and calculate adjusted hazard ratios. The Lifelines package was used to compute survival curves estimated using the Kaplan Meier method. All statistical tests were two-sided with a significance level of 2.5% for each tail. Assessment of classifier performance was done consistently for each analysis, using AUCs computed across the entire multimodal cohort by merging classifier scores obtained in the validation folds of 10-fold cross-validation. Model outputs were calibrated to an optimal threshold using the Youden Index and rescaled to a unit variance. The 95% confidence intervals on these AUCs were calculated using DeLong's methods for computing the covariance of unadjusted AUCs. Violins were smoothed using the default Gaussian kernel density estimation in seaborn v0.11.1. Significance of the AUCs were obtained against the hypothesis of meaningless class labels using perturbation testing with 20 iterations, with the covariance error and standard error of the mean propagated in quadrature.

TABLE 2
Model classification metrics. F1-Score, precision score, recall score, area
under curve (AUC), and accuracy of various models described herein.

| | | Precision | Recall | | |
|---|---|---|---|---|---|
| Model | F1-score | Score | Score | AUC | Accuracy |
| LR Clinical | 0.680982 | 0.787234 | 0.600000 | 0.569606 | 0.577236 |
| LR Rad-PC | 0.736364 | 0.810000 | 0.675000 | 0.639341 | 0.644172 |
| LR Rad-LN | 0.682927 | 0.717949 | 0.651163 | 0.626938 | 0.611940 |
| LR Rad-Average | 0.722892 | 0.796460 | 0.661765 | 0.645040 | 0.631016 |
| MILR Rad-Lesions | 0.632479 | 0.755102 | 0.544118 | 0.620314 | 0.540107 |
| LR IHC-A | 0.666667 | 0.701754 | 0.634921 | 0.623961 | 0.619048 |
| LR IHC-G | 0.666667 | 0.722222 | 0.619048 | 0.633787 | 0.628571 |
| LR PDL1-TPS | 0.788530 | 0.839695 | 0.743243 | 0.729284 | 0.706468 |
| LR Path-A-Average | 0.802817 | 0.838235 | 0.770270 | 0.695117 | 0.721393 |
| LR Path-G-Average | 0.811189 | 0.840580 | 0.783784 | 0.694225 | 0.731343 |
| LR Gen-Only-TMB | 0.769231 | 0.817143 | 0.772973 | 0.606931 | 0.700405 |
| LR Gen-No-TMB | 0.769231 | 0.924242 | 0.329730 | 0.605100 | 0.477733 |
| LR Gen-Average | 0.655462 | 0.813253 | 0.729730 | 0.658195 | 0.672065 |
| LR Gen-Combined | 0.716511 | 0.813253 | 0.729730 | 0.654795 | 0.672065 |
| DyAM IHC-A | 0.773946 | 0.696429 | 0.619048 | 0.605631 | 0.609524 |
| DyAM Gen | 0.755245 | 0.845588 | 0.621622 | 0.675850 | 0.631579 |
| DyAM Rad | 0.777448 | 0.808000 | 0.742647 | 0.701413 | 0.684492 |
| DyAM Rad + IHC-A | 0.755245 | 0.812030 | 0.705882 | 0.680753 | 0.668246 |
| DyAM IHC-A + Gen | 0.777448 | 0.861842 | 0.708108 | 0.714996 | 0.696356 |
| DyAM Rad + Gen | 0.785924 | 0.858974 | 0.724324 | 0.759285 | 0.704453 |
| DyAM TMB + PDL1 | 0.746269 | 0.833333 | 0.675676 | 0.693112 | 0.655870 |
| DyAM Rad + IHC-A + Gen | 0.783626 | 0.853503 | 0.724324 | 0.754228 | 0.700405 |
| DyAM Rad + IHC-G + GEN | 0.794118 | 0.870968 | 0.729730 | 0.782563 | 0.716599 |
| DyAM Rad + IHC-A + | 0.818713 | 0.891720 | 0.756757 | 0.798169 | 0.748988 |
| Gen + PDL1 | | | | | |
| DyAM Rad + IHC-G + | 0.836676 | 0.890244 | 0.789189 | 0.783348 | 0.769231 |
| Gen + PDL1 | | | | | |
| LR Multimodal-Average | 0.777778 | 0.847134 | 0.718919 | 0.723365 | 0.692308 |

### B. Systems and Methods of Determining Predicted Response Scores of Subjects Using Multimodal Models

A subject suffering from a condition (e.g., any type of cancer) may be evaluated to determine whether a therapy (e.g., immunotherapy) would be effective in treating the condition. A computing platform may be used to assist a clinician examining the subject to make the determination using measurements obtained from the subject, such as those derived from gene sequencing data (e.g., tumor mutation burden) or an immunohistochemistry image (e.g., PD-L1 score). The reliance on a single modality of data from the subject, however, may yield poor diagnosis and inaccurate predictions regarding receptiveness of the subject to immunotherapy. Furthermore, the computing platform may have to rely on separate applications to process different modalities of data. This may lead to wasted computing resources (e.g., processor and memory) from processing the data to provide poor and inaccurate results.

To address these and other technical challenges, a computing system may use a machine learning (ML) model to take inputs from multiple modalities to produce accurate predicted likelihood that the given subject would respond to the immunotherapy. The modalities may include, for example, data derived from immunohistochemistry images, radiological scans, and genomic sequencing of the subject. The ML model may include attention gates or weights (e.g., structured in accordance with deep attention-based multiple-instance learning model with masking (DyAM)) trained using data annotated by domain experts (e.g., clinicians or pathologists) to extract and weigh discriminative features. By applying this ML model, the computing system may produce more accurate and useful results regarding subject response to immunotherapy in a quicker manner. From providing the more accurate results, the computing system may save resources that would have otherwise been spent on producing less useful results. Furthermore, the computing system may lessen user burden from performing analyses on the different modalities of data separately, with the amalgamation of such data into the ML model.

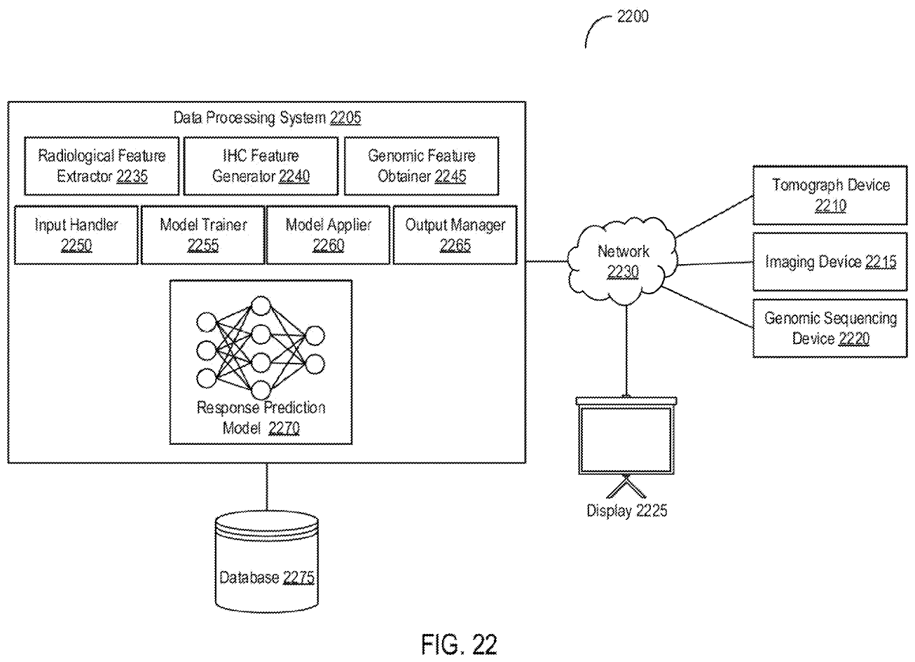

Referring now to FIG. 22, depicted is a block diagram of a system 2200 for determining risk scores using multimodal feature sets. In overview, the system 2200 may include at least one data processing system 2205, at least one tomograph device 2210, at least one imaging device 2215, at least one genomic sequencing device 2220, and at least one display 2225, communicatively coupled via at least one network 2230. The data processing system 2205 may include at least one radiological feature extractor 2235, at least one histological feature acquirer 2240, at least one genomic feature obtainer 2245, at least one input handler 2250, at least one model trainer 2255, at least one model applier 2260, and at least one output handler 2265, at least one response prediction model 2270, and at least one database 2275, among others. Each of the components in the system 2200 as detailed herein may be implemented using hardware (e.g., one or more processors coupled with memory), or a combination of hardware and software as detailed herein in Section C. Each of the components in the system 2200 may implement or execute the functionalities detailed herein, such as those described in Section A.

The data processing system 2205 may (sometimes herein generally referred to as a computing system or a service) be any computing device comprising one or more processors coupled with memory and software and capable of performing the various processes and tasks described herein. The data processing system 2205 may be in communication with the one or more of the tomograph device 2210, the imaging device 2215, and the genomic sequencing device 2220 via the network 2230. The data processing system 2205 may be situated, located, or otherwise associated with at least one server group. The server group may correspond to a data center, a branch office, or a site at which one or more servers corresponding to the data processing system 2205 is situated.

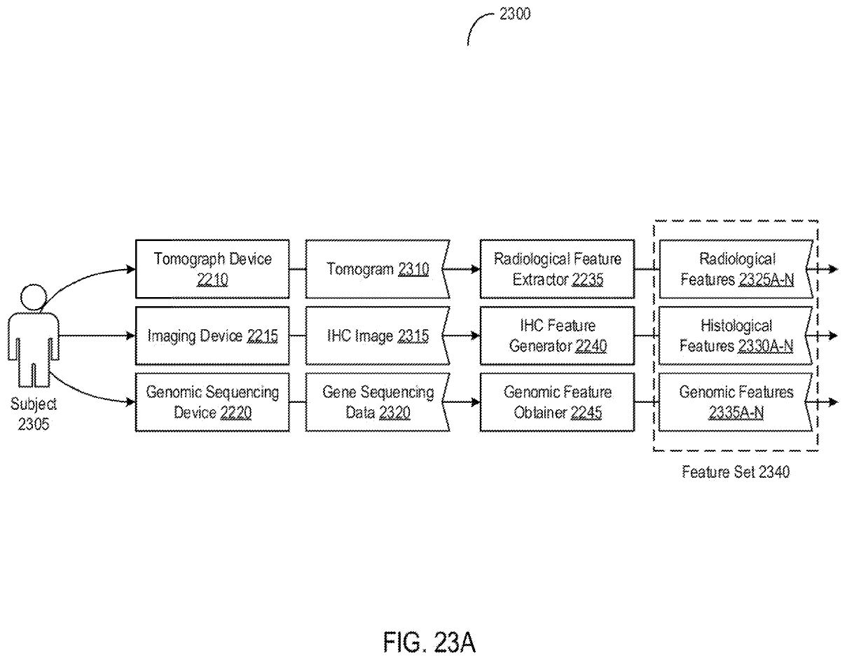

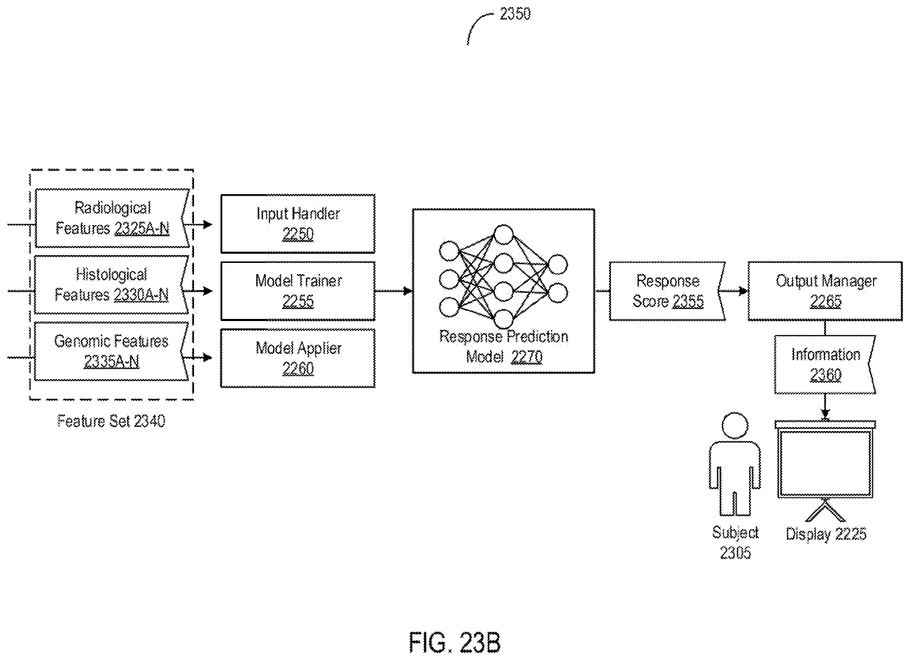

Referring now to FIG. 23A, depicted is a block diagram of a process 2300 of extracting multimodal features in the system 2200 for determining predicted responses. The process 2300 may correspond to or include operations in the system 2200 for identifying features in various modalities from subjects. Under the process 2300, one or more devices of the system 2200 may obtain or acquire data in multiple modalities from at least a portion of a subject 2305 (e.g., a human or animal). The subject 2305 may be suffering from, may be afflicted with, or may otherwise have a condition 2310 on at least one location in a body of the subject 2305. In some embodiments, the subject 2305 may be a risk of the condition 2310. The condition 2310 may include, for example, a type of cancer (e.g., breast cancer, bladder cancer, cervical cancer, colorectal cancer, kidney cancer, liver cancer, lung cancer, lymphoma, ovarian cancer, prostate cancer, skin cancer, or thyroid cancer), among others.

The subject 2305 may be under evaluation to determine whether to administer a treatment (e.g., immunotherapy) to address the condition 2310. The immunotherapy may include, for example: an immune checkpoint inhibitor such as programmed cell death protein 1 (PD-1) (e.g., Pembrolizumab, Nivolumab, Cemiplimab), Programmed death-ligand 1 (PD-L1) (e.g., Atezolizumab, Avelumab, Durvalumab), and cytotoxic T-lymphocyte-associated protein 4 (CTLA-4) (e.g., Ipilimumab), among others; an adoptive cell therapy, such as tumor-infiltrating lymphocyte (TIL) therapy, engineered T-cell receptor (TCR) therapy, and chimeric antigen receptors (CARs) cell therapy, among others; monoclonal antibodies (e.g., naked, conjugated, and bispecific); immune system modulator (e.g., interleukins, interferons, and immunomodulatory); oncolytic virus therapy; and cancer vaccines, among others.

The tomograph device 2210 may produce, output, or otherwise generate at least one tomogram 2315 (sometimes herein referred to generally as a biomedical image or an image) of a section of the subject 2305. The section may be associated with the condition 2310. For example, the tomogram 2315 may be a scan of the sample corresponding to a tissue of the organ in the subject 2305 affected by the type of cancer. The tomogram 2315 may include a set of two-dimensional cross-sections (e.g., a front, a sagittal, a transverse, or an oblique plane) acquired from the three-dimensional volume. The tomogram 2315 may be defined in terms of pixels, in two-dimensions or three-dimensions. In some embodiments, the tomogram 2315 may be part of a video acquired of the sample over time. For example, the tomogram 2315 may correspond to a single frame of the video acquired of the sample over time at a frame rate.

The tomogram 2315 may be acquired using any number of imaging modalities or techniques. For example, the tomogram 2315 may be a tomogram acquired in accordance with a tomographic imaging technique, such as a magnetic resonance imaging (MRI) scanner, a nuclear magnetic resonance (NMR) scanner, X-ray computed tomography (CT) scanner, an ultrasound imaging scanner, and a positron emission tomography (PET) scanner, and a photoacoustic spectroscopy scanner, among others. The tomogram 2315 may be a single instance of acquisition (e.g., X-ray) in accordance with the imaging modality, or may be part of a video (e.g., cardiac MRI) acquired using the imaging modality.

The tomogram 2315 may include or identify at least one at least one region of interest (ROI) (also referred herein as a structure of interest (SOI) or feature of interest (FOI)). The ROI may correspond to an area, section, or part of the tomogram 2315 that corresponds to the presence of the condition 2310 in the sample from which the tomogram 2315 is acquired. For example, the ROI may correspond to a portion of the tomogram 2315 depicting a tumorous growth in a CT scan of a brain of a human subject. With the acquisition of the tomogram 2315, the tomograph device 2210 may send, transmit, or otherwise provide the tomogram 2315 to the data processing system 2205. The tomogram 2315 may be maintained using one or more files in accordance with a format (e.g., single-file or multi-file DICOM format).

The imaging device 2215 may scan, obtain, or otherwise acquire an immunohistochemistry (IHC) image 2320 (sometimes herein referred generally as a biomedical image or image) of a sample (e.g., the sample) of the subject 2305. The sample may be obtained from the section of the subject 2305 used to generate the IHC image 2320, or may be taken from another portion associated with the condition 2310 within the subject 2305. The IHC image 2320 may be acquired or derived using immunostaining techniques (e.g., immunofluorescence) in accordance with a staining modality. The staining modality may correspond to stain selected to identify a particular antigen, protein, or other biomarker in the sample from the subject 2305. The biomarkers may include inhibitors for the immunotherapy, such as PD-1, PD-L1, and CTLA-4, among others.

The IHC image 2320 may include one or more regions of interest (ROIs). Each ROI may correspond to portions within the sample IHC image 2320 associated with the condition 2310. For example, the ROIs depicted in the IHC image 2320 may correspond to areas with cell nuclei associated with particular antigen, protein, or other biomarkers for the type of cancer. The staining modality may be used to differentiate the ROI from remaining portions of the IHC image 2320. The IHC image 2320 may be maintained using one or more files in accordance with a format (e.g., DICOM). Upon generation, the imaging device 2215 may send, transmit, or otherwise provide the IHC image 2320 to the data processing system 2205.

The genomic sequencing device 2220 may carry out, execute, or otherwise perform genetic sequencing on a deoxyribonucleic acid (DNA) sample taken from the subject 2305 to generate gene sequencing data 2325. The genetic sequencing carried out may be a high throughput, massively parallel sequencing technique (sometimes herein referred to as next generation sequencing), such as pyrosequencing, Reversible dye-terminator sequencing, SOLiD sequencing, Ion semiconductor sequencing, Helioscope single molecule sequencing, among others.

The genetic sequencing performed by the genomic sequencing device 2220 may be targeted to find biomarkers associated with or correlated with the condition 2310 of the subject 2305. For example, for lung cancer, the genomic sequencing device 2220 may perform the targeted sequencing to find an altered oncogene (e.g., epidermal growth factor receptor (EFGR), anaplastic lymphoma kinase (ALK), encoded receptor tyrosine kinase (ROS1), rearranged during transfection (RET) gene, human epidermal growth factor receptor (HER2) or receptor tyrosine-protein kinase erbB-2 (ERBB2), BRAF, and MET gene); altered tumor suppressors (e.g., Serine/threonine kinase 11 (STK11); and altered transcription regulators (e.g., AT-rich interactive domain-containing protein 1A (ARID1A)), among others. Upon carrying out the sequencing, the genomic sequencing device 2220 may send, transmit, or otherwise provide the gene sequencing data 2325 to the data processing system 2205. The gene sequencing data 2325 may be maintained using one or more files according to a format (e.g., FASTQ, BCL, or VCF formats).

The radiological feature extractor 2235 executing on the data processing system 2205 may generate, determine, or otherwise identify a set of radiological features 2330A-N (hereinafter generally referred to as radiological features 2330) using the tomogram 2315. The radiological features 2330 may include or identify information derived from the tomogram 2315. The radiological feature extractor 2235 may apply one or more machine learning (ML) models to derive, determine, or otherwise generate the radiological features 2330 from the tomogram 2315. The ML model may include a set of weights to process at least one input (e.g., the tomogram 2315) to generate at least one output (e.g., the set of radiological features 2330). The ML models may include, for example: an image segmentation model to determine the ROI within the tomogram 2315 associated with the condition 2310; an image classification model to determine the type of feature (e.g., type of tissue associated with the condition 2310) to which to classify sample depicted in the tomogram 2315; or an image localization model to determine a portion (e.g., a tile) within the tomogram 2315 corresponding to the ROI, among others. The ML model for image segmentation, localization, or classification may be of any architecture, such as a deep learning artificial neural network (ANN), a regression model (e.g., linear or logistic regression), a clustering model (e.g., k-NN clustering or density-based clustering), Naïve Bayesian classifier, a decision tree, a relevance vector machine (RVM), or a support vector machine (SVM), among others.

With the application of the ML model, the radiological feature extractor 2235 may determine or identify at least one segment of the tomogram 2315 corresponding to the ROI. The ROI may correspond to the condition 2310 in the scanned section of the subject 2305 from which the tomogram 2315 is acquired. With the identification of the segment corresponding to the ROI, the radiological feature extractor 2235 may determine, derive, or otherwise generate the set of radiological features 2330 (e.g., as listed above). In some embodiments, the radiological feature extractor 2235 may generate an initial set of radiological features 2330 using the original segment. In some embodiments, the radiological feature extractor 2235 may generate another set of radiological features 2330 using a perturbation of the segment. The perturbation may include, for example: noise addition, translation, rotation, volume modification (e.g., increase or decrease), and contour distortion, among others.

From the initial set of radiological features 2330, the radiological feature extractor 2235 may identify or select a reduced set of set of radiological features 2330 based on robustness measures. The radiological feature extractor 2235 may calculate, generate, or otherwise determine a robustness measure for each radiological feature 2330 in the initial set. The robustness measure may identify or correspond to a correlation of a given the radiological feature 2330 across the initial set and the perturbation set. For instance, the robustness measure may be calculated as a ratio of a variance across the perturbation set versus a variance across both sets. Using the robustness measures, the radiological feature extractor 2235 may select a subset of the initial set radiological features 2330 satisfying a cutoff threshold.

The resultant radiological features 2330 may include or identify information derived from the segment identified from the tomogram 2315 corresponding to the ROI. For example, the radiological features 2330 may identify or include one or more characteristics of a shape or a size of the ROI identified from the tomogram 2315, such as: an area (e.g., percentage) of the ROI within the tomogram 2315; or a dimension (e.g., diameter, length, or width along a given axis) of the given ROI; among others. The radiological features 2330 may also identify or include a descriptor of relationships among voxels, such as: an intensity histogram (IH), an intensity-volume histogram (IVH), a gray-level co-occurrence matrix (GLCM), a gray-level run-length matrix (GLRLM), a gray-level size zone matrix (GLSZM), a gray-level distance zone matrix (GLDZM), a neighborhood gray tone difference matrix (NGTDM), and a neighborhood gray tone dependence matrix (NGLDM), among others. The radiological features 2330 may include statistical measures of the descriptor, such as autocorrelation, skewness, kurtosis, mean, or variance, among others, of the IH, IVH, GLCM, GLRLM, GLSZM, GLDZM, NGTDM, and NGLDM.

The IHC feature generator 2240 executing on the data processing system 2205 may generate, determine, or otherwise identify a set of IHC features 2335A-N (hereinafter generally referred to as IHC features 2335) using the IHC image 2320. The radiological features 2330 may include or identify information derived from the from IHC image 2320. The IHC feature generator 2240 may apply one or more machine learning (ML) models to derive, determine, or otherwise generate the IHC features 2335 from the IHC image 2320. The ML model may include a set of weights to process at least one input (e.g., the IHC image 2320) to generate at least one output (e.g., the set of IHC features 2335).

The ML models may include, for example: an image segmentation model to determine the ROI within the IHC image 2320 associated with the condition; an image classification model to determine the condition type to which to classify sample depicted in the IHC image 2320; or an image localization model to determine a portion (e.g., a tile) within the IHC image 2320 corresponding to the ROI, among others. The ML model for image segmentation, localization, or classification may be of any architecture, such as a deep learning artificial neural network (ANN), a regression model (e.g., linear or logistic regression), a clustering model (e.g., k-NN clustering or density-based clustering), Naïve Bayesian classifier, a decision tree, a relevance vector machine (RVM), or a support vector machine (SVM), among others.

From applying the image segmentation or localization model, the IHC feature generator 2240 may determine a portion of the IHC image 2320 corresponding to the one or more ROIs. The ROI may correspond to cell nuclei associated with particular antigen, protein, or other biomarkers for the condition 2310. As discussed above, the biomarkers may include, for example, inhibitors for the immunotherapy, such as PD-1, PD-L1, and CTLA-4, among others. With the determination, the IHC feature generator 2240 may calculate, determine, or identify the set of IHC features 2335 to include one or more pixel-derived properties of the ROIs in the IHC image 2320, such as: an area (e.g., percentage) of the ROI within the IHC image 2320; or a dimension (e.g., diameter, length, or width along a given axis) of the given ROI in the IHC image 2320; or a statistical measure (e.g., mean, median, standard deviation) in staining (e.g., H&E) indicative of the tissue type or cell nuclei type; among others.

In some embodiments, the IHC feature generator 2240 may calculate, determine, or identify the set of IHC features 2335 to include a descriptor of relationships of the pixels within the IHC image 2320, such as: an intensity histogram (IH), an intensity-volume histogram (IVH), a gray-level co-occurrence matrix (GLCM), a gray-level run-length matrix (GLRLM), a gray-level size zone matrix (GLSZM), a gray-level distance zone matrix (GLDZM), a neighborhood gray tone difference matrix (NGTDM), and a neighborhood gray tone dependence matrix (NGLDM), among others. The IHC features 2335 may include statistical measures of the descriptor, such as autocorrelation, skewness, kurtosis, mean, or variance, among others, of the IH, IVH, GLCM, GLRLM, GLSZM, GLDZM, NGTDM, and NGLDM. The IHC features 2335, such as GLCM, may be correlated with a tumor proportion score (TPS) or a progression-free survival (PFS) measure as discussed in Section A.

The genomic feature obtainer 2245 executing on the data processing system 2205 may generate, determine, or otherwise identify a set of genomic features 2340A-N (hereinafter generally referred to genomic features 2340) using the gene sequencing data 2220. Using the gene sequencing data 2220, the genomic feature obtainer 2245 may identify or determine one or more genes associated with immunotherapy response for the condition 2310 as the set of genomic features 2340. For example, the genes for the genomic features 2340 may include an altered oncogene (e.g., epidermal growth factor receptor (EFGR), anaplastic lymphoma kinase (ALK), encoded receptor tyrosine kinase (ROS1), rearranged during transfection (RET) gene, human epidermal growth factor receptor (HER2) or receptor tyrosine-protein kinase erbB-2 (ERBB2), BRAF, and MET gene); altered tumor suppressors (e.g., Serine/threonine kinase 11 (STK11); and altered transcription regulators (e.g., AT-rich interactive domain-containing protein 1A (ARID1A)), among others.

With the identification, the radiological features 2330, the IHC features 2335, and genomic features 2340 may form at least one feature set 2345 (sometimes herein referred to as a multimodal feature set). The feature set 2345 may include one or more features from a variety of modalities, as described herein. In some embodiments, the feature set 2345 may lack at least one of the features, such as one or two of the radiological feature 2330, the IHC feature 2335, and genomic features 2340. The feature set 2345 may be further processed by the data processing system 2205 to evaluate the subject 2305. At least some of the feature sets 2340 together with expected response scores may be used for training the response prediction model 2270 as explained below. At least some of the feature sets 2345 may be used at runtime to feed to the response prediction model 2270 to determine predicted response scores for subjects 2305.

Referring now to FIG. 23B, depicted is a block diagram of a process 2350 of applying response prediction models to multimodal features. The process 2350 may correspond to or include operations in the system 2200 for establishing a multimodal model and determining response scores for subjects. Under the process 2350, the model trainer 2255 executing on the data processing system 2205 may initialize or establish the response prediction model 2270 (sometimes herein referred to as a multimodal or multivariate model). The model trainer 2255 may be invoked to establish the response prediction model 2270 during training mode. The response prediction model 2270 may be any machine learning (ML), such as: a regression model (e.g., linear or logistic regression), a clustering model (e.g., k-NN clustering or density-based clustering), Naïve Bayesian classifier, artificial neural network (ANN), a decision tree, a relevance vector machine (RVM), or a support vector machine (SVM), among others. For example, the response prediction model 2270 may have an architecture described herein in Section A, such as the description of the dynamic deep attention-based multiple-instance learning model with masking (DyAM) in conjunction with FIG. 5A. In general, the response prediction model 2270 may have one or more inputs corresponding to the feature set 2345, one or more outputs for predicted response scores, and one or more weights (also referred herein as attention weights) relating the inputs and the outputs, among others.

To establish the response prediction model 2270, the model trainer 2255 may retrieve, receive, or identify training data. The training data may include a set of examples, and may be maintained on the database 2275. Each example may include or correspond to a feature set 2345 and corresponding expected response score. Each feature set 2345 may identify or include one or more of the radiological features 2330, the histological features 2335, and genomic features 2340 for a given sample subject 2305. Each expected (or measured) response score may correspond to or identify whether the subject 2305 responded to the immunotherapy to treat or address the condition 2310. The immunotherapy associated with the expected response score may include, for example, immune checkpoint inhibitor, an adoptive cell therapy, monoclonal antibodies, immune system modulator, oncolytic virus therapy, or cancer vaccines, among others.

In some embodiments, the expected response score may measure, correspond to, or otherwise identify a likelihood of responding to the immunotherapy when administered to the subject 2305. For example, the expected response score may measure how likely the subject 2305 is to improve or be treated when provided with the immunotherapy. The expected response score may be manually created by a clinician examining the subject 2305 from which the feature set 2345 is obtained. In some embodiments, the training data may identify or include the expected response score over a period of time. The period of time may range, for example, from 3 days to 5 years. The model trainer 2255 may set the weights of the response prediction model 2270 to initial values (e.g., zero or random) when initializing.

From the training data, the input handler 2250 executing on the data processing system 2205 may extract or identify the feature set 2345 for training the response prediction model 2270. The feature set 2345 may identify or include one or more of the radiological features 2330, the IHC features 2335, and the genomic features 2340, among others. For some examples in the training data, the feature set 2345 may lack at least one of the radiological features 2330, the IHC features 2335, and the genomic features 2340. For instance, one or two of the radiological features 2330, the IHC features 2335, or the genomic features 2340 may be unavailable for a given subject 2305. In some examples, one of the features for a given modality in the feature set 2345 may be missing or otherwise unavailable. For instance, the IHC features 2335 may include the skewness of the GCLM autocorrelation matrix but lack the dimensions of the ROI in the IHC image 2320. Whereas in other examples, the feature set 2345 may include all of the radiological features 2330, the IHC features 2335, and the genomic features 2340 for the subject 2305.

With the identification, the input handler 2250 may identify or determine whether at least one of the features (e.g., the radiological features 2330, the IHC features 2335, and the genomic features 2340) of the feature set 2345 is unavailable or missing. To determine, the input handler 2250 may parse the feature set 2345 to find, extract, or identify the features therein. If all are available, the input handler 2250 may determine that the all of the features of the feature set 2345 are available. With this determination, the input handler 2250 may maintain the features of the feature set 2345 with the initial values. On the other hand, if any are unavailable, the input handler 2250 may determine that at least one of the features in the feature set 2345 is unavailable. From the feature set 2345, the input handler 2250 may identify the one or more feature is unavailable. The input handler 2250 may set or assign each feature identified as unavailable to a defined value (e.g., null or another random value). By assigning the feature to the defined value, the input handler 2250 may perform or carry out a masking function for the response prediction model 2270. The input handler 2250 may traverse over the examples of the training data to identify each feature set 2345 for training the response prediction model 2270.

The model applier 2260 may apply the identified feature set 2345 to the response prediction model 2270. To apply, the model applier 2260 may feed the feature set 2345 into the input of the response prediction model 2270. In some cases, the feature set 2345 may include one feature assigned to the defined value, upon determination that the feature (e.g., the radiological feature 2330, the IHC feature 2335, and the genomic features 2340) is unavailable. From feeding, the model applier 2260 may process the input in accordance with the set of weights of the response prediction model 2270 to produce, output, or otherwise generate a predicted response score for the feature set 2345. The predicted response score may be similar to the expected response score and may identify the likelihood of responding to the immunotherapy when administered to the subject 2305 to address the condition. The immunotherapy associated with the predicted response score 2355 may include, for example, immune checkpoint inhibitor, an adoptive cell therapy, monoclonal antibodies, immune system modulator, oncolytic virus therapy, or cancer vaccines, among others. In some embodiments, the output may include the survival function defining or identifying the predicted response score over the period of time. The survival function may identify, specify, or otherwise define a set of values for the predicted response score at a given set of time instances (e.g., at defined intervals) in the time period.

In determining the overall predicted response score, the model applier 2260 may calculate, generate, or otherwise determine a set of constituent risk scores for each feature in the feature set 2345 using at least a portion of the weights of the response prediction model 2270 applied to the features. For instance, the model applier 2345 may generate a constituent risk score for the radiological features 2330, another constituent risk score for the IHC features 2335, and constituent risk score for the genomic features 2340. To generate, the model applier 2260 may select or identify a subset of weights of the response prediction model 2270 to be applied to the feature for the given modality (e.g., radiological, IHC, and genomic). The subset of weights may correspond to weights of at least one layer of the response prediction model 2270 towards the inputs. With the identification, the model applier 2260 may process or apply the subset of weights of the response prediction model 2270 to the corresponding feature. From applying, the model applier 2260 may generate the constituent risk score for the feature.

Using the set of constituent risk scores, the model applier 2260 may calculate, determine, or otherwise generate the predicted response score for the subject 2305. To generate, the model applier 2260 may select or identify a remaining set of weights of the response prediction model 2270. The remaining set of weights may be in a layer of the response prediction model 2270 by the output and may succeed from or be subsequent to the weights used to calculate the set of constituent risk scores. For instance, the model applier 2260 may feed the set of constituent risk scores from the weights of the input layer toward the weights of the output layer in the response prediction model 2260. The model applier 2260 may feed or process the set of constituent risk scores in accordance with the weights of the output layer to generate the predicted response score for the subject 2305. With the generation of the predicted response score 2355, the model applier 2260 may relay, convey, or otherwise provide the predicted response score 2355 to the model trainer 2255 under the training mode.

Upon the generation of the output from the response prediction model 2270, the model trainer 2255 may compare the predicted response score with the expected response score from the same example with the feature set 2345. Based on the comparison, the model trainer 2255 may update the weights of the response prediction model 2270. In some embodiments, the model trainer 2255 may calculate, generate, or otherwise determine at least one loss metric (sometimes herein referred to as an error metric) based on the comparison. The loss metric may identify or correspond to a degree of deviation of the predicted risk score from the expected risk score. The loss metric may be calculated in accordance with any number of loss functions, such as a mean squared error (MSE), a mean absolute error (MAE), a hinge loss, a quantile loss, a quadratic loss, a smooth mean absolute loss, and a cross-entropy loss, among others. Using the loss metric, the model trainer 2255 may update the weights of the response prediction model 2270. The updating (e.g., backpropagation in accordance with an optimization function with a learning rate) of the weights of the response prediction model 2270 may be repeated until reaching convergence as defined for the model architecture.

With the training and establishment of the response prediction model 2270, the input handler 2250 may retrieve, receive, or otherwise identify newly acquired data. The newly acquired data may correspond to or include the feature set 2345 when received during a runtime mode (also referred herein as an evaluation mode). The values of the features in the features set 2345 may differ from those used during training mode. The feature set 2345 may include radiological features 2330, the IHC features 2335, and the genomic features 2340, among others. The functionalities of the model applier 2260 and input handler 2250 under runtime mode may be similar to the functionalities of the model applier 2260 and input handler 2250 under training mode as discussed above.

From the acquired data, the input handler 2250 may extract or identify the feature set 2345 to apply to the response prediction model 2270. The feature set 2345 may identify or include one or more of the radiological features 2330, the IHC features 2335, and the genomic features 2340, among others. For some newly acquired data, the feature set 2345 may lack at least one of the radiological features 2330, the IHC features 2335, and the genomic features 2340. For instance, one or two of the radiological features 2330, the IHC features 2335, or the genomic features 2340 may be unavailable for a given subject 2305. In some examples, one of the features for a given modality in the feature set 2345 may be missing or otherwise unavailable. Whereas in other examples, the feature set 2345 may include all of the radiological features 2330, the IHC features 2335, and the genomic features 2340 for the subject 2305.

With the identification, the input handler 2250 may identify or determine whether at least one of the features (e.g., the radiological features 2330, the IHC features 2335, and the genomic features 2340) of the feature set 2345 is unavailable or missing. To determine, the input handler 2250 may parse the feature set 2345 to find, extract, or identify the features therein. If all are available, the input handler 2250 may determine that the all of the features of the feature set 2345 are available. With this determination, the input handler 2250 may maintain the features of the feature set 2345 with the initial values. On the other hand, if any are unavailable, the input handler 2250 may determine that at least one of the features in the feature set 2345 is unavailable. From the feature set 2345, the input handler 2250 may identify the one or more feature is unavailable. The input handler 2250 may set or assign each feature identified as unavailable to a defined value (e.g., null or another random value). By assigning the feature to the defined value, the input handler 2250 may perform or carry out a masking function for the response prediction model 2270.

The model applier 2260 may apply the identified feature set 2345 to the response prediction model 2270. To apply, the model applier 2260 may feed the feature set 2345 into the input of the response prediction model 2270. In some cases, the feature set 2345 may include one feature assigned to the defined value (e.g., null or another value), upon determination that the feature (e.g., the radiological feature 2330, the IHC feature 2335, and the genomic features 2340) is unavailable. From feeding, the model applier 2260 may process the input in accordance with the set of weights of the response prediction model 2270 to produce, output, or otherwise generate a predicted response score 2355 for the feature set 2345. The predicted response score 2355 may be similar to the expected response score and may identify the likelihood of responding to the immunotherapy when administered to the subject 2305 to address the condition. In some embodiments, the output may include the survival function defining or identifying the predicted response score 2355 over the period of time. The survival function may identify, specify, or otherwise define a set of values for the predicted response score 2355 at a given set of time instances (e.g., at defined intervals) in the time period.

In determining the overall predicted response score 2355, the model applier 2260 may calculate, generate, or otherwise determine a set of constituent risk scores for each feature in the feature set 2345 using at least a portion of the weights of the response prediction model 2270 applied to the features. For instance, the model applier 2345 may generate a constituent risk score for the radiological features 2330, another constituent risk score for the IHC features 2335, and constituent risk score for the genomic features 2340. To generate, the model applier 2260 may select or identify a subset of weights of the response prediction model 2270 to be applied to the feature for the given modality (e.g., radiological, IHC, and genomic). The subset of weights may correspond to weights of at least one layer of the response prediction model 2270 towards the inputs. With the identification, the model applier 2260 may process or apply the subset of weights of the response prediction model 2270 to the corresponding feature. From applying, the model applier 2260 may generate the constituent risk score for the feature.

Using the set of constituent risk scores, the model applier 2260 may calculate, determine, or otherwise generate the predicted response score 2355 for the subject 2305. To generate, the model applier 2260 may select or identify a remaining set of weights of the response prediction model 2270. The remaining set of weights may be in a layer of the response prediction model 2270 by the output and may succeed from or be subsequent to the weights used to calculate the set of constituent risk scores. For instance, the model applier 2260 may feed the set of constituent risk scores from the weights of the input layer toward the weights of the output layer in the response prediction model 2260. The model applier 2260 may feed or process the set of constituent risk scores in accordance with the weights of the output layer to generate the predicted response score 2355 for the subject 2305. With the generation of the predicted response score 2355, the model applier 2260 may relay, convey, or otherwise provide the predicted response score 2355 to the output handler 2265 under the runtime mode.

With the generation of the output from the response prediction model 2270, the output manager 2265 executing on the data processing system 2205 may generate an association between the predicted response score 2355 and the subject 2305 using one or more data structures, such as a linked list, a tree, an array, a table, a matrix, a stack, a queue, or a heap, among others. In some embodiments, the association may be among the predicted risk scores 2265, the set of constituent risk scores, the subject 2305 (e.g., using an anonymized identifier), the condition 2310, the features of the feature set 2345, and data used to generate the feature set 2340 (e.g., the tomogram 2315, the IHC image 2320, and gene sequencing data 2325), among others. The data structures for the association may be stored and maintained on the database 2270.

In some embodiments, the output manager 2265 may categorize, assign, or otherwise classify the subject 2305 into one of a set of response level groups (sometimes herein referred to as a response cohort) based on the predicted response score 2355. The groups may be used to classify subjects 2305 by predicted response score 2355. For example, one group may correspond to a low likelihood that the subject 2305 would respond to the type of immunotherapy associated with the predicted response score 2355. Another group may correspond to a high likelihood that the subject 2305 would respond to the type of immunotherapy. To classify, the output handler 2265 may compare the predicted response score 2355 for the subject 2305 with a threshold for each response level group. The threshold may delineate or define a value (or range) for the predicted risk score 2355 above which the subject 2305 is to be classified into the associated response level group. When the predicted response score 2355 satisfies the threshold for at least one response level group, the output handler 2265 may assign the subject 2305 (e.g., using the anonymized identifier) to the associated response level group. The output handler 2265 may store the association of the response level group with the subject 2305 on the database 2270 using the one or more data structures.

In some embodiments, the output manager 2265 may produce, create, or otherwise generate information 2360 based on the predicted response score 2355 (or the association). The information 2360 may include instructions for rendering, displaying, or otherwise presenting the predicted response score 2355, along with the identifier for the subject 2305 and the feature set 2345, among others. Upon generation, the output handler 2265 may send, transmit, or otherwise provide the information 2360 to the display 2225 (or a computing device coupled with the display 2225). The provision of the information 2360 may be in response to a request from a user of the data processing system 2205 or the computing device. The display 2225 may render, display, or otherwise present the information 2360, such as the predicted response score 2355, the feature set 2340, and the identifier for the subject 2305, among others. For example, the computing device may execute an application to process the information 2360. The application running on the computing device may display, render, or otherwise present a graphical user interface with the information 2360, such as the predicted response score 2360, constituent risk scores, and the response group for the subject 2305, adjacent to the tomogram 2315, the IHC images 2320, and the gene sequencing data 2325 for the subject 2305.

In this manner, the data processing system 2205 may process the feature set 2345 derived from data of different modalities (e.g., the tomogram 2315, the IHC image 2320, and the genomic sequencing data 2325) thereby widening the types of data used to apply to the response prediction model 2270. Even with the absence or unavailability of certain features, the response prediction model 2270 may be trained using annotations by domain experts (e.g., clinicians or pathologists) to extract and weigh discriminative features. With the establishment of the response prediction model 2270, the data processing system 2205 may process and generate the predicted response score 2355 in a more meaningful and accurate manner, relative to other techniques. By processing with the response prediction model 2270, the data processing system 2205 may be able to computing resources (e.g., the processor and memory) that would have otherwise been spent on producing less useful outputs. Furthermore, the data processing system 2205 may lessen user burden from performing analyses on the features of different modalities separately, with the amalgamation of such data into the response prediction model 2270.

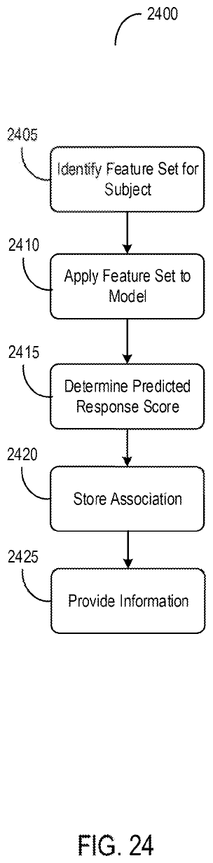

Referring now to FIG. 24, depicted is a flow diagram of a method 2400 of determining predicted response using multimodal feature sets. The method 2400 may be performed by or implementing using the system 2200 described herein in conjunction with FIGS. 22-23B or the system 2500 as described herein in conjunction with Section C. Under the method 2400, a computing system (e.g., the data processing system 2205) may identify a feature set (e.g., the feature set 2345) for a subject (e.g., the subject 2305) (2405). The computing system may apply the feature set to a model (e.g., the response prediction model 2270) (2410). The computing system may a predicted response score (e.g., the response score 2355) from application of the model (2415). The computing system may store an association between the predicted response score and the subject (2420). The computing system may provide information (e.g., the information 2360) based on the output (2425).

### C. Computing and Network Environment

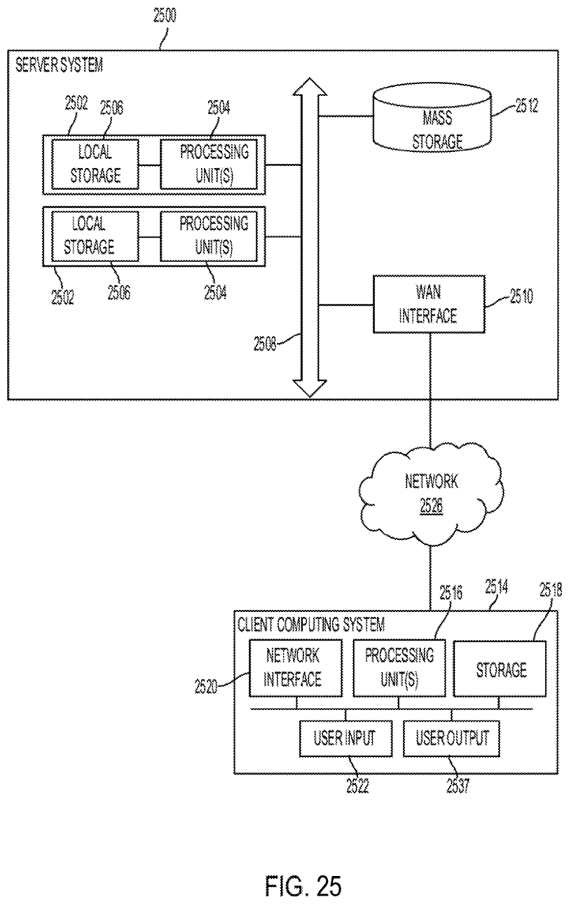

Various operations described herein can be implemented on computer systems. FIG. 25 shows a simplified block diagram of a representative server system 2500, client computer system 2514, and network 2526 usable to implement certain embodiments of the present disclosure. In various embodiments, server system 2500 or similar systems can implement services or servers described herein or portions thereof. Client computer system 2514 or similar systems can implement clients described herein. The systems 2200 described herein can be similar to the server system 2500. Server system 2500 can have a modular design that incorporates a number of modules 2502 (e.g., blades in a blade server embodiment); while two modules 2502 are shown, any number can be provided. Each module 2502 can include processing unit(s) 2504 and local storage 2506.

Processing unit(s) 2504 can include a single processor, which can have one or more cores, or multiple processors. In some embodiments, processing unit(s) 2504 can include a general-purpose primary processor as well as one or more special-purpose co-processors such as graphics processors, digital signal processors, or the like. In some embodiments, some or all processing units 2504 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 2504 can execute instructions stored in local storage 2506. Any type of processors in any combination can be included in processing unit(s) 2504.

Local storage 2506 can include volatile storage media (e.g., DRAM, SRAM, SDRAM, or the like) and/or non-volatile storage media (e.g., magnetic or optical disk, flash memory, or the like). Storage media incorporated in local storage 2506 can be fixed, removable, or upgradeable as desired. Local storage 2506 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 2504 need at runtime. The ROM can store static data and instructions that are needed by processing unit(s) 2504. The permanent storage device can be a non-volatile read-and-write memory device that can store instructions and data even when module 2502 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

In some embodiments, local storage 2506 can store one or more software programs to be executed by processing unit(s) 2504, such as an operating system and/or programs implementing various server functions such as functions of the systems 2200 or any other system described herein, or any other server(s) associated with systems 2200 or any other system described herein.

“Software” refers generally to sequences of instructions that, when executed by processing unit(s) 2504, cause server system 2500 (or portions thereof) to perform various operations, thus defining one or more specific machine embodiments that execute and perform the operations of the software programs. The instructions can be stored as firmware residing in read-only memory and/or program code stored in non-volatile storage media that can be read into volatile working memory for execution by processing unit(s) 2504. Software can be implemented as a single program or a collection of separate programs or program modules that interact as desired. From local storage 2506 (or non-local storage described below), processing unit(s) 2504 can retrieve program instructions to execute and data to process in order to execute various operations described above.

In some server systems 2500, multiple modules 2502 can be interconnected via a bus or other interconnect 2508, forming a local area network that supports communication between modules 2502 and other components of server system 2500. Interconnect 2508 can be implemented using various technologies including server racks, hubs, routers, etc.

A wide area network (WAN) interface 2510 can provide data communication capability between the local area network (interconnect 2508) and the network 2526, such as the Internet. Technologies can be used, including wired (e.g., Ethernet, IEEE 802.3 standards) and/or wireless technologies (e.g., Wi-Fi, IEEE 802.11 standards).

In some embodiments, local storage 2506 is intended to provide working memory for processing unit(s) 2504, providing fast access to programs and/or data to be processed while reducing traffic on interconnect 2508. Storage for larger quantities of data can be provided on the local area network by one or more mass storage subsystems 2512 that can be connected to interconnect 2508. Mass storage subsystem 2512 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 2512. In some embodiments, additional data storage resources may be accessible via WAN interface 2510 (potentially with increased latency).

Server system 2500 can operate in response to requests received via WAN interface 2510. For example, one of the modules 2502 can implement a supervisory function and assign discrete tasks to other modules 2502 in response to received requests. Work allocation techniques can be used. As requests are processed, results can be returned to the requester via WAN interface 2510. Such operation can generally be automated. Further, in some embodiments, WAN interface 2510 can connect multiple server systems 2500 to each other, providing scalable systems capable of managing high volumes of activity. Other techniques for managing server systems and server farms (collections of server systems that cooperate) can be used, including dynamic resource allocation and reallocation.

Server system 2500 can interact with various user-owned or user-operated devices via a wide-area network such as the Internet. An example of a user-operated device is shown in FIG. 25 as client computing system 2514. Client computing system 2514 can be implemented, for example, as a consumer device such as a smartphone, other mobile phone, tablet computer, wearable computing device (e.g., smart watch, eyeglasses), desktop computer, laptop computer, and so on.

For example, client computing system 2514 can communicate via WAN interface 2510. Client computing system 2514 can include computer components such as processing unit(s) 2516, storage device 2518, network interface 2520, user input device 2522, and user output device 2537. Client computing system 2514 can be a computing device implemented in a variety of form factors, such as a desktop computer, laptop computer, tablet computer, smartphone, other mobile computing device, wearable computing device, or the like.

Processing unit(s) 2516 and storage device 2518 can be similar to processing unit(s) 2504 and local storage 2506 described above. Suitable devices can be selected based on the demands to be placed on client computing system 2514; for example, client computing system 2514 can be implemented as a “thin” client with limited processing capability or as a high-powered computing device. Client computing system 2514 can be provisioned with program code executable by processing unit(s) 2516 to enable various interactions with server system 2500.

Network interface 2520 can provide a connection to the network 2526, such as a wide area network (e.g., the Internet) to which WAN interface 2510 of server system 2500 is also connected. In various embodiments, network interface 2520 can include a wired interface (e.g., Ethernet) and/or a wireless interface implementing various RF data communication standards such as Wi-Fi, Bluetooth, or cellular data network standards (e.g., 3G, 4G, LTE, etc.).

User input device 2522 can include any device (or devices) via which a user can provide signals to client computing system 2514; client computing system 2514 can interpret the signals as indicative of particular user requests or information. In various embodiments, user input device 2522 can include any or all of a keyboard, touch pad, touch screen, mouse or other pointing device, scroll wheel, click wheel, dial, button, switch, keypad, microphone, and so on.

User output device 2537 can include any device via which client computing system 2514 can provide information to a user. For example, user output device 2537 can include display-to-display images generated by or delivered to client computing system 2514. The display can incorporate various image generation technologies, e.g., a liquid crystal display (LCD), light-emitting diode (LED) including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that function as both input and output device. In some embodiments, other user output devices 2537 can be provided in addition to or instead of a display. Examples include indicator lights, speakers, tactile “display” devices, printers, and so on.

Some embodiments include electronic components, such as microprocessors, storage, and memory that store computer program instructions in a computer readable storage medium. Many of the features described in this specification can be implemented as processes that are specified as a set of program instructions encoded on a computer readable storage medium. When these program instructions are executed by one or more processing units, they cause the processing unit(s) to perform various operations indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 2504 and 2516 can provide various functionality for server system 2500 and client computing system 2514, including any of the functionality described herein as being performed by a server or client, or other functionality.

It will be appreciated that server system 2500 and client computing system 2514 are illustrative and that variations and modifications are possible. Computer systems used in connection with embodiments of the present disclosure can have other capabilities not specifically described here. Further, while server system 2500 and client computing system 2514 are described with reference to particular blocks, it is to be understood that these blocks are defined for convenience of description and are not intended to imply a particular physical arrangement of component parts. For instance, different blocks can be but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

While the disclosure has been described with respect to specific embodiments, one skilled in the art will recognize that numerous modifications are possible. Embodiments of the disclosure can be realized using a variety of computer systems and communication technologies, including, but not limited to, specific examples described herein. Embodiments of the present disclosure can be realized using any combination of dedicated components and/or programmable processors and/or other programmable devices. The various processes described herein can be implemented on the same processor or different processors in any combination. Where components are described as being configured to perform certain operations, such configuration can be accomplished; e.g., by designing electronic circuits to perform the operation, by programming programmable electronic circuits (such as microprocessors) to perform the operation, or any combination thereof. Further, while the embodiments described above may refer to specific hardware and software components, those skilled in the art will appreciate that different combinations of hardware and/or software components may also be used and that particular operations described as being implemented in hardware might also be implemented in software or vice versa.

Computer programs incorporating various features of the present disclosure may be encoded and stored on various computer readable storage media; suitable media includes magnetic disk or tape, optical storage media such as compact disk (CD) or digital versatile disk (DVD), flash memory, and other non-transitory media. Computer readable media encoded with the program code may be packaged with a compatible electronic device, or the program code may be provided separately from electronic devices (e.g., via Internet download or as a separately packaged computer-readable storage medium).

Thus, although the disclosure has been described with respect to specific embodiments, it will be appreciated that the disclosure is intended to cover all modifications and equivalents within the scope of the following claims.

What is claimed is:

1. A method of determining predicted response scores of subjects, comprising:
identifying, by a computing system, a first feature set for a first subject to be administered with immunotherapy to address a condition, the first feature set comprising one or more of:
(i) a first radiological feature identified in a tomogram of a section associated with the condition in the first subject,
(ii) a first immunohistochemistry (IHC) feature derived from an image of a sample associated with the first subject, and
(iii) a first genomic feature obtained from gene sequencing of the first subject for genes associated with the condition,
applying, by the computing system, the first feature set to a model comprising a set of weights, wherein the set of weights for the model is established using (i) a plurality of second feature sets from a respective plurality of second subjects and (ii) a plurality of expected scores each identifying a respective response to immunotherapy in corresponding second subject of the plurality of second subjects;
determining, by the computing system, from applying the first feature set to the model, a predicted score identifying a response to the immunotherapy to be administered to the first subject; and
storing, by the computing system, using one or more data structures, an association between the first subject and the predicted score identifying the response.

2. The method of claim 1, further comprising:
determining, by the computing system, that at least one feature of the first feature set corresponding to the first radiological feature, the first IHC feature, and the first genomic feature is unavailable; and
assigning, by the computing system, a defined value to the at least one feature in the first feature set, responsive to determining that the at least one feature is unavailable, and
wherein applying the first feature set further comprises applying the first feature set comprising the at least one feature assigned to the defined value.

3. The method of claim 1, further comprising:
determining, by the computing system, that all of features corresponding to the first radiological feature, the first IHC feature, and the first genomic feature of the first feature set are available; and
maintaining, by the computing system, the first feature set responsive to determining that all the features in the first feature set are available.

4. The method of claim 1, further comprising classifying, by the computing device, the first subject into one of a plurality of response groups based on a comparison between the predicted score identifying a likelihood of improvement from the immunotherapy and a threshold for each of the plurality of response groups.

5. The method of claim 1, wherein determining the predicted score further comprises determining a plurality of risk scores for the predicted score, the plurality of risk scores identifying: (i) a first score corresponding to the first radiological feature, (ii) a second score corresponding to the first IHC feature, and (iii) the first genomic feature.

6. The method of claim 1, wherein determining the predicted score further comprises generating a survival function identifying the predicted score for the response to the immunotherapy by the first subject over a time period.

7. The method of claim 1, wherein the first radiological feature is based on a region of interest (ROI) identified in the tomogram corresponding to a portion of the section associated with the condition to be addressed with the immunotherapy.

8. The method of claim 1, wherein the first IHC feature derived from the image is based on a gray level co-occurrence matrix (GLCM) autocorrelation matrix correlated with at least one of a tumor proportion score (TPS) or a progression-free survival (PFS) measure.

9. The method of claim 1, wherein the first genomic feature identifies one or more genes associated with therapy response comprising at least one of: (i) an altered oncogene, (ii) an altered tumor suppressor, or (iii) an altered transcription regulator.

10. The method of claim 1, further comprising providing, by the computing system, information based on the association between the first subject and the predicted score identifying the response.

11. A system for determining predicted responses of subjects to treatments, comprising:
a computing system having one or more processors coupled with memory, configured to:
identify a first feature set for a first subject to be administered with immunotherapy to address a condition, the first feature set comprising one or more of:
(i) a first radiological feature identified in a tomogram of a section associated with the condition in the first subject,
(ii) a first immunohistochemistry (IHC) feature derived from an image of a sample associated with the first subject, and
(iii) a first genomic feature obtained from gene sequencing of the first subject for genes associated with the condition,
apply the first feature set to a model comprising a set of weights, wherein the set of weights for the model is established using (i) a plurality of second feature sets from a respective plurality of second subjects and (ii) a plurality of expected scores each identifying a respective response to immunotherapy in corresponding second subject of the plurality of second subjects;
determine, from applying the first feature set to the model, a predicted score identifying a response to the immunotherapy to be administered to the first subject; and
store, using one or more data structures, an association between the first subject and the predicted score identifying the response.

12. The system of claim 11, wherein the computing system is further configured to:
determine that at least one feature of the first feature set corresponding to the first radiological feature, the first IHC feature, and the first genomic feature is unavailable; and
assign a defined value to the at least one feature in the first feature set, responsive to determining that the at least one feature is unavailable, and
apply the first feature set comprising the at least one feature assigned to the defined value.

13. The system of claim 11, wherein the computing system is further configured to
determine that all of features corresponding to the first radiological feature, the first IHC feature, and the first genomic feature of the first feature set are available; and
maintain the first feature set responsive to determining that all the features in the first feature set are available.

14. The system of claim 11, wherein the computing system is further configured to classify, the first subject into one of a plurality of response groups based on a comparison between the predicted score identifying a likelihood of improvement from the immunotherapy and a threshold for each of the plurality of response groups.

15. The system of claim 11, wherein the computing system is further configured to determine a plurality of risk scores for the predicted score, the plurality of risk scores identifying: (i) a first score corresponding to the first radiological feature, (ii) a second score corresponding to the first IHC feature, and (iii) the first genomic feature.

16. The system of claim 11, wherein the computing system is further configured to generate a survival function identifying the predicted score for the response to the immunotherapy by the first subject over a time period.

17. The system of claim 11, wherein the first radiological feature is based on a region of interest (ROI) identified in the tomogram corresponding to a portion of the section associated with the condition to be addressed with the immunotherapy.

18. The system of claim 11, wherein the first IHC feature derived from the image is based on a gray level co-occurrence matrix (GLCM) autocorrelation matrix correlated with at least one of a tumor proportion score (TPS) or a progression-free survival (PFS) measure.

19. The system of claim 11, wherein the first genomic feature identifies one or more genes associated with therapy response comprising at least one of: (i) an altered oncogene, (ii) an altered tumor suppressor, or (iii) an altered transcription regulator.

20. The system of claim 11, wherein the computing system is further configured to provide information based on the association between the first subject and the predicted score identifying the response.
